(19) United States
(12) Patent Application Publication
(10) Pub. No.: US 2025/0281441 A1
(43) Pub. Date: Sep. 11, 2025

(54) ORODISPERSIVE DOSAGE FORMS CONTAINING DROXIDOPA

(71) Applicant: APRECIA PHARMACEUTICALS LLC, Mason, OH (US)

(72) Inventors: Kelly E. CAPUTO, Langhorne, PA (US); Kaoru Maeda, East Brunswick, NJ (US); Thomas G. West, Lawrenceville, NJ (US); Amol S. Matharu, East Windsor, NJ (US); Lauren E. Beach-Herrera, East Brunswick, NJ (US); Thomas J. Bradbury, Yardley, PA (US); Jaedeok Yoo, Princeton, NJ (US)

(21) Appl. No.: 19/210,673

(22) Filed: May 16, 2025

Related U.S. Application Data
(63) Continuation of application No. PCT/US2023/080208, filed on Nov. 17, 2023.
(60) Provisional application No. 63/426,691, filed on Nov. 18, 2022.
(60) Provisional application No. 63/598,507, filed on Nov. 13, 2023.

(51) Int. Cl. A61K 31/198; A61K 9/00; A61K 47/02; A61K 47/10; A61K 47/12; A61K 47/18; A61K 47/22; A61K 47/26; A61K 47/32; A61K 47/38

(52) U.S. Cl. CPC A61K 31/198; A61K 9/0056; A61K 47/02; A61K 47/10; A61K 47/12; A61K 47/183; A61K 47/22; A61K 47/26; A61K 47/32; A61K 47/38

(57) ABSTRACT

A rapidly-orodispersible tablet containing a porous, bound powder matrix containing droxidopa. The bound matrix contains an acidulant, particularly citric acid, to delay, reduce, or inhibit the coloration of droxidopa during stress storage conditions, and suppress the effect of glycerin on the coloration of droxidopa-containing tablets. The tablet can be dividable along a functional seam printed into the bound powder matrix, to divide the tablet into sub-dosage units of the same mass of droxidopa.

### CROSS-REFERENCE TO EARLIER FILED APPLICATIONS

The present application is a continuation of International Application No. PCT/US2023/080208 filed Nov. 17, 2023, which claims the benefits of U.S. Provisional Application No. 63/426,691 filed Nov. 18, 2022, and U.S. Provisional Application No. 63/598,507 filed Nov. 13, 2023, the entire disclosures of which are hereby incorporated by reference.

### FIELD OF THE INVENTION

The present invention relates in general to the field of manufacturing of dosage forms comprising droxidopa.

### BACKGROUND OF THE INVENTION

Droxidopa is an orally active synthetic precursor of norepinephrine and is converted to norepinephrine in the body by aromatic L-amino acid decarboxylase (AAAD), also known as DOPA decarboxylase (DDC), to replenish depleted norepinephrine, allow for re-uptake into neurons of the peripheral nervous system, and provide physiological improvement in symptomatic neurogenic orthostatic hypotension patients. Droxidopa has also been studied or approved for the treatment of frozen gait or dizziness associated with Parkinson's disease, as well as depression, hemodialysis induced vertigo, dizziness and weakness, syncope or dizziness associated with Shy-Drager syndrome, and familial amyloidotic polyneuropathy.

Chemically, the form of droxidopa commonly administered to patients is L-threo-dihydroxyphenylserine (also known in the pharmaceutical industry as DOPS, threo-DOPS, and L-DOPS). While droxidopa is optically active and can be provided in various forms, including L-threo-DOPS, D-threo-DOPS, L-erythro-DOPS, and D-erythro-DOPS, L-threo enantiomer is generally recognized to be the desired isomer having desired activity. The chemical structure of L-DOPS is illustrated below.

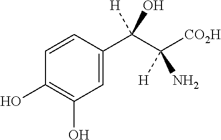 [SMILES: N[C@H](C(=O)O)[C@H](O)c1ccc(O)c(O)c1]

Tablet forms of droxidopa are generally easiest to administer (see, e.g., U.S. Pat. No. 8,980,316, the disclosure of which is herein incorporated by reference in its entirety). For example, Droxidopa is currently available in the market as Northera® capsules, with dosages of 100 mg, 200 mg, and 300 mg for oral administration. Starting dose is 100 mg three times during the day, up to a maximum dose of 600 mg three times daily (1800 mg daily). However, tablets and capsules can be difficult to administer to some patients, particularly patients who are older, are affected with Parkinson's disease, and/or otherwise have trouble swallowing medication.

As a result, there is an interest within the marketplace to develop droxidopa dosage forms that are easier for a patient to self-administer, such as orally disintegrating tablets (“ODT”), which quickly disintegrate in a patient's oral cavity without causing discomfort. In one non-limiting example, U.S. Pat. No. 8,980,316 (hereinafter, the '316 patent), the disclosure of which is incorporated by reference in its entirety, describes compressed tablets comprising droxidopa and bound by corn-derived starch and/or polyvinyl alcohol, which preferably disintegrate within 80 seconds of being placed inside an oral cavity. Yet, the compressed droxidopa tablets were shown to change color when mixed with certain excipients, under humid conditions, and at elevated temperatures, requiring the use of dry capsules (such as Northera®) to better protect the droxidopa from the surrounding environment. Although the '316 patent identified a handful of excipients to avoid using when forming compressed droxidopa tablets, the best results arose from storing the completed tablets in sealed, brown bottles in the presence of a silica gel desiccate, which led to, on average, a 2.5-fold improvement over samples stored without the desiccate (see e.g., Table 2 of the '316 Patent). Consequently, there remains a need to develop droxidopa formulations and dosage forms with an increased resistance to color change that are not dependent on storage conditions.

ODTs produced by conventional compressed-tableting techniques are inherently limited with respect to the mass of an active pharmaceutical ingredient (API) that can be contained within a single tablet, as a balance must be struck between compressing the dosage forms enough to for them to remain intact as a tablet, while also not compressing them so tightly that the tablets are no longer rapidly orodispersible. Further, it is believed that when a compressed ODT gets too large, the tablet interior becomes shielded from solvent (e.g., saliva) until the tablet periphery disintegrates, increasing the disintegration time to the point where it is no longer rapid. As an example, the '316 patent describes tablets having, at most, 200 mg of droxidopa out of a 400-mg total mass, and the highest dose of any active ingredient approved by Food and Drug Administration (FDA) to be contained within a compressed ODT is 275 mg (PARCOPA®).

As an alternative, three-dimensional printing (3DP) has been shown to be useful in the manufacture of rapidly-orodispersible dosage forms. Such doses include amounts of the API above 300 mg per tablet. Non-limiting examples of three-dimensionally printed (3DP) rapidly-orodispersible dosage forms are described in U.S. Pat. Nos. 9,314,429, 9,339,489, and 9,492,380, the disclosures of which are incorporated by reference in their entireties.

Therefore, it would be useful to develop dosage forms comprising droxidopa that are resistant to color change, which can contain up to 300 mg or more of droxidopa, and/or that are rapidly-orodispersible, preferably disintegrating within a person's oral cavity within 30 seconds.

### SUMMARY OF THE INVENTION

The present invention describes dosage forms generally comprising (a) one or more monoamines and/or their precursors in particulate form, and (b) a binder, in which the dosage form may be prescribed to a patient to treat a disease or disorder that is therapeutically responsive to the one or more monoamines and/or their precursors. The monoamines described herein have substantially similar chemical and physical properties, namely a common structural scaffold comprising a single amino group connected to an aromatic ring by a two-carbon chain, and generally derive from precursors synthesized via biosynthetic pathways in which the essential aromatic amino acids phenylalanine, tyrosine, and tryptophan are modified by one or more hydrolases and/or decarboxylases.

In various embodiments, the one or more monoamines can comprise any combination of monoamine neuromodulators and/or monoamine transmitters. In various embodiments, one or more of the monoamine neuromodulators can be selected from the group consisting of: phenylethylamine, N-methylphenethylamine, m-tyramine, p-tyramine, N-methyltyramine, m-octopamine, p-octopamine, synephrine, 3-methoxytyramine, phenylethanolamine, thyronamine, 3-iodothyonamine, tryptamine, and any combination, precursor, or derivative thereof. In various embodiments, one or more of the monoamine transmitters can belong to a class of compounds selected from the group of: an imadazoleamine, a non-limiting example of which is histamine; a catecholamine, non-limiting examples of which are adrenaline (or alternatively, “epinephrine”), dopamine, and noradrenaline (or alternatively, “norepinephrine”); an indolamine, non-limiting examples of which are serotonin and melatonin, and any combination, precursor, or derivative thereof.

In various embodiments, the dosage form comprises one or more catecholamines and/or natural or synthetic catecholamine precursors in particulate form. In some embodiments, a catecholamine precursor can be a compound selected from the group consisting of droxidopa, L-DOPA, p-tyramine, L-tyrosine, L-phenylalanine, and any combination thereof. In some embodiments, a dosage form of the present invention comprising droxidopa can be administered to a patient as a pro-drug to norepinephrine. In some embodiments, a dosage form of the present invention comprising L-DOPA can be administered to a patient as a pro-drug to epinephrine.

In various embodiments, droxidopa is the primary, or sole, active pharmaceutical ingredient (hereinafter, “API”) within any of the dosage forms described herein.

Unless otherwise noted, within any of the embodiments described below in which droxidopa is the API, any one or more of the monoamines or monoamine precursors described herein can be substituted in place of droxidopa, without limitation or departing from the scope of the invention. In one non-limiting example, L-DOPA can be substituted in place of droxidopa in any of the embodiments described herein and below. Other examples and embodiments in which another monoamine or monoamine precursor is substituted in place of droxidopa or L-DOPA are omitted for both brevity and clarity.

The present invention provides a rapidly orodispersible tablet <1> comprising a porous, bound matrix comprising: up to about 66% droxidopa, about 3% to about 35% of a disintegrant, up to about 5% of an acidulant, up to about 5% glycerin, and a binder, wherein the bound matrix comprises droxidopa particles bound by the binder, has a porosity of about 30% to about 80%, and disperses in 30 seconds or less in a volume of 30 ml or less of water or saliva. In some embodiments, acidulant comprises an organic acid, and the organic acid can comprise citric acid. In some embodiments, the binder is a water-soluble binder.

In some embodiments, the dosage form is a rapidly-orodispersible tablet comprising a porous, bound powder matrix, the bound powder matrix formed from a build powder and a binding liquid. Forming the rapidly-orodispersible tablets is preferably accomplished using a three-dimensional printing (3DP) process, particularly a binder jetting process comprising the following steps: providing a blend of particulate components as an unbound build powder; forming the unbound build powder into a layer having a generally uniform thickness; dispensing a binding liquid from a print head onto the unbound powder layer to form a wetted matrix; and removing excess liquid from (or drying) the wetted matrix to form a bound matrix comprising the particulate blend and non-volatile components of the binding liquid.

The present invention also provides an orodispersible tablet <2> comprising about 20% to about 80% droxidopa, about 3% to about 35% of a disintegrant, up to about 5% of an acidulant, and a binder, wherein the tablet comprises a bound matrix comprising droxidopa particles bound by the binder, and disperses in 30 seconds or less in a volume of 30 ml or less of water or saliva. In some embodiments, the binder is a water-soluble binder.

In various embodiments, a dosage form comprises from about 1 mg to about 5,000 mg droxidopa. Non-limiting examples of the mass of droxidopa within a dosage form are at least about 100 mg, at least about 200 mg, at least about 300 mg, at least about 400 mg, and at least about 600 mg, and up to about 1,800 mg, up to about 1,200 mg, and up to about 900 mg. In various embodiments, a dosage form comprises any one of about 100 mg, about 200 mg, about 300 mg, about 400 mg, or about 600 mg droxidopa. The dosage form typically comprises, or is made using, droxidopa in particle form, having a particle size from about 1 μm to about 1 mm.

In various embodiments, droxidopa is present within the dosage forms as particulate particles having a particle size of at least about 1 μm and less than about 1 mm. In some embodiments, droxidopa particles can be provided in their native form as a substantially pure drug substance for use as a build powder in a 3DP process. In some embodiments, droxidopa particles can be provided for a build powder as a pre-blend of droxidopa powder material dry blended with one or more excipients. In some embodiments, droxidopa particles can be provided for a build powder as granules of droxidopa formed using any granulation technique recognized in the relevant arts (e.g., including without limitation, fluid bed granulation or high shear granulation), or combinations thereof. In some embodiments, the droxidopa particles provided for the build powder can have a particle size distribution such that their D(10) is at least about 1 μm and up to about 150 μm, their D(50) is at least about 5 μm and up to about 350 μm, and/or their D(90) is at least about 10 μm and up to about 600 μm.

Droxidopa particles, as well as freshly-manufactured dosage forms containing droxidopa, are generally a white or an off-white color. However, droxidopa is known to be susceptible to chemical aging, particularly by oxidation, when the droxidopa is exposed to moisture, elevated temperatures, UV light, and/or certain chemical compounds (see e.g., U.S. Pat. No. 8,980,316, above). As a result, particles, compositions, and dosage forms comprising droxidopa can change color as they age, particularly in higher temperatures and humidity, and can transition from white or off-white to yellow, to brown, and/or to black over time. Similar phenomena have also been observed with L-DOPA, carbidopa, dopamine, epinephrine, tyrosine, and tryptophan.

Accordingly, and in various embodiments, dosage forms comprising particulate droxidopa or another monoamine and/or monoamine precursor can also comprise an acidulant to delay, reduce, and/or inhibit color development or color change as compared to the dosage form without the acidulant. In various embodiments, the acidulant is distributed substantially uniformly throughout the bound matrix that comprises the droxidopa particulate and other particulate excipients bound together with the binder.

The acidulant can be highly dispersed throughout the dosage form and/or distributed throughout the bound matrix as particles. In some embodiments, the acidulant is dispersed throughout the bound powder matrix as fine particulate matter (about 2.5 micrometers or less) or as ultrafine particulate matter (about 0.1 micrometers or less).

In some embodiments, acidulent compound can be dispersed molecularly (essentially as a non-particulate) when delivered as a dissolved component in the binding liquid used to bind the build powder into the bound matrix. In such embodiments, the binding liquid containing the dissolved acidulant is dispersed as fine (nano- and/or micro-scale) droplets into the build powder, and contacts and wets the particulate droxidopa and other particulate excipients of the build powder. Some of the particulate components of the build powder can be partially dissolved by, and be dispersed into, the binding liquid, to mix with the binder, the dissolved acidulant, and other excipients of the binding liquid. Without being bound by any particular theory, when an excess of the solvent, which can include water and other solvents, in the printed and distributed binding liquid evaporates from the wetted bound matrix, the dissolved particulate, the dissolved acidulant, and other non-volatile components of the binding liquid, remain dispersed and become distributed essentially molecularly within and throughout the wetted bound matrix, including across and in direct contact with the outer surfaces of, and between, the API particles, such as the droxidopa particles, and the other bound powder particulate.

In some embodiments, the acidulant or a portion of the acidulant is dispersed throughout the bound matrix as particulate acidulant, typically provided as a particulate component of the build powder, which is then formed with the other particulate components, including particulate droxidopa and/or other APIs, of the build powder into a bound powder matrix. On an equivalent mass basis in a dosage form, the total number of acidulant particles distributed through the bound matrix is inversely proportional to their particle size (individual particle mass), generally by the third power. Consequently, the smaller the particle size, such as average particle size, the greater the number of particles that are distributed within a unit volume of the bound matrix or along a unit surface of a particulate component of the bound matrix, including along a unit surface of a droxidopa particle. Without being bound by any particular theory, it is believed that increasing the number of acidulant particles across a unit surface of a droxidopa particle improves the performance of the acidulant in delaying, reducing, and/or inhibiting color development or color change of the droxidopa particles. In some embodiments, when acidulant, particularly anhydrous citric acid, is introduced into the bound matrix as a particulate, the acidulant can have a D(90) of at least about 10, 25, 50, 100, 150, 200, 250, 300, 400, or 500 μm and/or up to about 600, 500, 400, 300, 250, 200, 150, 100, 50, or 25 μm, and for example a D(90) of citric acid particles of up to about 400, or about 200, or about 125, or about 100, or about 75 μm, and/or at least about 70, or about 100, or about 150 μm.

On the other hand, an increasingly smaller particle size of a component of the build powder can increase the risk of separation or segregation of such smaller-sized particles from the remaining particulate of the build powder, which can occur during storage or transport of the build powder to the dosage-forming process equipment. Persons of ordinary skill in the art of powder blending and handling can evaluate particulate acidulant of various average particle sizes in a particular tablet-forming process equipment and determine a target range of particle size of the acidulant that avoids or sufficiently minimize acidulant particulate segregation, to provide effective coloration performance in the dosage form while maintaining processability and target acidulant levels in the build powder.

The acidulant comprises an organic acid selected from the group consisting of aspartic acid, citric acid, fumaric acid, glutamic acid, malic acid, maleic acid, malonic acid, oxalic acid, oxaloacetic acid, succinic acid, tartaric acid, and a combination thereof. In some embodiments, the acidulant comprises, consists essentially of, or consists of, citric acid.

In some embodiments, the dosage form can comprise an acidulant at a percent by mass of at least about 0.1%, 0.3%, 0.5%, 0.7%, 0.9%, 1.0%, 1.25%, 1.5%, 1.75%, 2.0%, 2.5%, 3.0%, 3.5%, 4.0%, or 4.5%, and/or up to about 5.0%, 4.5%, 4.0%, 3.5%, 3.0%, 2.5%, 2.0%, 1.75%, 1.5%, 1.25%, 1.0%, 0.9%, 0.7%, 0.5%, or 0.3%, or from about 0.1% to about 0.5%, or from about 0.5% to about 1.0%.

In various embodiments, any of the dosage forms described herein can comprise at least about 5% and up to about 80%, of a binder, wherein the binder can comprise one or more binder materials. In a binder jetting process, binder materials can be processed into the tablet as particulates blended uniformly into the build powder, or as a dissolved component within the binding liquid, or a combination thereof. In some embodiments, the binder comprises at least one particulate material in the build powder and/or at least one dissolved binder material within the binding liquid. In some embodiments, the binder is water soluble.

In various embodiments, a dosage form comprises a binder in an amount sufficient and effective to bind the build powder into a bound powder matrix that has sufficient hardness and durability to retain its form and content during ordinary handling conditions. In some embodiments, a dosage form can comprise excess binder beyond an amount sufficient and effective to bind the build powder into the bound powder matrix. In further embodiments, at least a portion of the excess binder can be utilized as a filler material to balance the total mass content of a dosage form after masses of the droxidopa, disintegrant, acidulant, and any other optional excipients have been selected.

In various embodiments, the binder comprises one or more of hydroxypropyl cellulose, povidone, copovidone, and mannitol. In various embodiments, the binder comprises a binder material selected from the group consisting of polyvinylpyrrolidone, poly (l-vinylpyrrolidone-co-vinyl acetate, hydroxypropyl methylcellulose, hydroxypropyl cellulose, agar, gelatin, and combinations or mixtures thereof, and in some embodiments, selected from the group consisting of hydroxypropyl cellulose, povidone, copovidone, mannitol, and any combination thereof. In some embodiments, the binder comprises at least about 1%, and up to about 75%, of mannitol as a filler material.

In various embodiments, any of the dosage forms described herein can comprise from about 3% to about 35%, and particularly from about 5% to about 15%, of a disintegrant. In some embodiments, the disintegrant comprises one or more disintegrant compounds selected from the group consisting of microcrystalline cellulose, cross-linked polyvinylpyrrolidone, croscarmellose, sodium starch glycolate, and a combination thereof. In some embodiments, the disintegrant comprises or consists of microcrystalline cellulose. In a binder jetting process, disintegrant compounds can be processed into the tablet as particulates blended uniformly into the build powder, to minimize contact with moisture prior to forming the dosage form.

In various embodiments, any of the dosage forms described herein can optionally comprise glycerin, a commonly-used excipient in pharmaceutical dosage forms that can be used as a humectant, sweetener, preservative, lubricant, saponifier, or solvent. In some embodiments, the dosage form can comprise up to about 5% of glycerin, with non-limiting examples of up to about 2%, or from about 0.1% to about 1.0%, or from about 0.5% to about 0.75%.

However, as described in the examples below, initial formulations of binder-jetted dosage forms comprising droxidopa and glycerin developed color at an accelerated rate relative to dosage forms prepared without glycerin. Accordingly, and in various embodiments, the mass of the acidulant can be controlled to delay, reduce, and/or inhibit the accelerated coloration of the droxidopa when glycerin is also present. In some embodiments, the mass ratio of acidulant to glycerin can be from about 1:10 to about 10:1, which can include a mass ratio of about 1:1, about 1:2, about 1:3, about 1:4, about 1:5, about 1:6, about 2:1, about 3:1, about 4:1, about 5:1, or about 6:1, and for example, from 1:2 to 1:7, or from 1:3 to 1:6.

In various embodiments, tablets <1> or <2> are prepared according to a 3DP binder-jetting process, and at least a portion of the acidulant is molecularly dispersed throughout the bound matrix <3>, and/or comprises particles of acidulant that are distributed throughout the bound matrix <4>, and/or the acidulant comprises citric acid <5>, and/or the tablet comprises up to about 2% acidulant, for example, citric acid <6>, and/or the acidulant particles, for example citric acid particles, have a D(90) value of up to about 100 microns <7>.

In various embodiments of any of the tablets <1> through <7>, the tablet comprises glycerin <8>, and/or up to 5%, and preferably up to 2%, glycerin <9>, and/or the glycerin is molecularly dispersed throughout the bound matrix <10>, and/or a mass ratio of the acidulent, for example citric acid, to the glycerin is from about 1:3 to about 1:6<11>.

In various embodiments of any of the tablets <1> through <11>, the tablet has a porosity of about 50% to about 70%<12>, and/or the water-soluble binder comprises a compound selected from the group consisting of polyvinylpyrrolidone, poly (l-vinylpyrrolidone-co-vinyl acetate, hydroxypropyl methylcellulose, hydroxypropyl cellulose, agar, gelatin, and combinations or mixtures thereof <13>, and/or the tablet comprises up to about 60% mannitol <14>, and/or the tablet comprises at least one excipient selected from the group consisting of one or more surfactants, one or more antioxidants, one or more glidants, one or more flavorants, one or more chelating agents, and one or more preservatives, and a combination thereof <15>.

In various embodiments of any of the tablets <1> through <15>, the droxidopa particles provided to the 3DP process via the build powder have a particle size distribution with a D(90) of about 200 microns or less <16>, and/or the tablet comprises 300 mg or more of droxidopa <17>.

In various embodiments of any of the tablets <1> through <17>, the tablet is dividable along one or more functional seams into two or more subunits having substantially equivalent mass with a mass tolerance of about 3.0% or less, including about 1.5% or less<18>, and/or the bound matrix comprises at least five layers of porous, bound powder bonded together by the water-soluble binder <19>, and/or the tablet has a hardness of at least about 0.5 kilopond (kp), preferably at least about 1 kp, and more preferably at least about 2 kp<20>.

According to the present invention, the dosage forms can be formulated as tablets comprising droxidopa, a binder, a disintegrant, an acidulant, and optionally, one or more additional pharmaceutically-acceptable excipients, non-limiting examples of which are dispersants, sweeteners, glidants, flavoring agents, surfactants, humectants, preservatives, chelating agents, antioxidants, fillers, lubricants, solvents, saponifiers, and diluents. In some embodiments, the tablet further comprises glycerin.

In various embodiments, the dosage forms described herein, and particularly rapidly-orodispersible 3DP tablets comprising droxidopa, can be manufactured using a binder jetting system, as an ingestible porous, interconnected matrix of particles of powder material and a binder material, using high-throughput continuous, semi-continuous, or batch manufacturing techniques, with minimal product loss, high efficiency, and high product reproducibility. In various embodiments, the dosage forms made using a binder jetting process can comprise a plurality of incremental printed layers. In another embodiment, the number of printed incremental layers can be from 3 layers to 50 layers, which can be from 5 layers to 15 layers.

In various embodiments, the dosage forms have an overall porosity from 1% to 90%, which can be at least from 5%, or at least from 10%, or at least from 20%, or at least from 40%, or at least from 50%, and up to 80%, or up to 70%. Preferably, the porosity of the dosage form is about 50% to 70%, for example about 60%.

In another embodiment, the bound powder matrix can have a bulk density from about 0.3 grams per cubic centimeter (g/cm³) to about 1.2 g/cm³; for example, from about 0.4 g/cm³ to about 0.8 g/cm³, or about 0.5-0.7 g/cm³, for example about 0.6 g/cm³.

In another embodiment, the bound powder matrix can have a hardness, as determined by a tablet breaking force assay according to USP <1217>, of any value, range, or sub-range between and inclusive of about 0.5 kilopond (kp) and about 20 kp. Non-limiting examples of such hardness ranges are at least about 1 kp, or at least 2 kp, or at least 3 kp; or from about 1 kp to about 5 kp; or from about 2 kp to about 8 kp.

As a non-limiting example, and in some embodiments, any of the 3DP droxidopa dosage forms described herein can be manufactured as a dividable tablet having one or more functional seams, which can be split to form two or more sub-dosage units. In various embodiments, the dosage form has a single functional seam that bisects the tablet and can be broken along the functional seam to form two sub-dosage units. In various embodiments, the two sub-dosage units have a substantially equivalent mass, within a difference between the two sub-dosage units being less than about 3%, 2%, 1.5%, 1.0%, 0.8%, 0.6%, or 0.5%. In some embodiments, each of the sub-dosage units comprise a body having a boundary wall, a periphery, and an interior body portion.

In various embodiments, a functional seam of any of the dividable 3DP tablets described herein comprises a first bound powder matrix, and the breakage-resistant boundary wall of each subunit comprises a second bound powder matrix. In some embodiments, the porosity of the first bound powder matrix is higher than the porosity of the second bound powder matrix. In some embodiments, the first bound powder matrix of the functional seam has a porosity that is at least 10% higher, and up to 50% higher, than the porosity of the second bound powder matrix of the boundary wall; for example, at least 15%, and up to 25% higher, or at least 15%, and up to 20% higher. The first bound powder matrix of the functional seam having the higher porosity comprises at least 50%, and preferably at least 90%, and up to 100%, of the planar structure of the functional seam.

Some of the dosage forms comprise a bound powder matrix, the bound powder matrix including at least two sub-dosage units and a functional seam, each of the at least two sub-dosage units including a boundary wall having a break strength, wherein the boundary walls of the at least two sub-dosage units confront one another, and are joined along opposite sides of and to the functional seam that has a break strength that is less than the break strength of the boundary walls. Applying a bending force on opposite sides of a stabilized functional seam will cause the tablet to subdivide along the functional seam without breaking the boundary walls.

In some embodiments, the break strength of a bound matrix or a coherent portion of a bound matrix is inversely proportional to its porosity, and the porosity of a portion of the bound matrix is inversely proportional to the quantity of the binding liquid dispensed by printing nozzles onto the layer of build powder when forming the bound powder matrix.

In various embodiments, a dividable tablet containing droxidopa comprises two or more sub-dosage units joined separably or dividably by a functional seam formed within the bound powder matrix. The dividable tablet has a total mass of droxidopa and can have a bisecting functional seam for dividing and separating the two sub-dosage units, which each contain half of the droxidopa total mass. The dividable tablet can be formed having three or more equal sub-dosage units and two or more functional seams, for dividing the dividable tablet into the three or more subunits that each contain a respective equal third or other fraction of the droxidopa total mass.

In various embodiments, any of the dividable 3DP tablets described herein can optionally include a physical score line, such as a groove or gully, in an outer surface of the tablet and along an axis of the functional seam. In other embodiments, the one or more functional seams of any of the dividable 3DP tablets described herein can be provided without a physical score line.

In various embodiments, for any of the dividable tablets described herein, the dividable tablets and respective divided subunits comply with some or all of the criteria for functional scoring described in U.S. FDA's Guidance for Industry-Tablet Scoring: Nomenclature, Labeling, and Data for Evaluation March 2013, hereby incorporated by reference in its entirety. In various embodiments, for any of the dividable tablets described herein, the tablet subunits optionally can demonstrate adequate stability for a period of 90 days at 25° C., plus or minus 2° C. and 60 percent Relative Humidity (RH), plus or minus 5 percent RH, when stored in pharmacy dispensing containers (no seal/no desiccant). In various embodiments, for any of the dividable tablets described herein, the tablet subunits optionally can meet the same finished-product testing requirements as for a whole-tablet product with equivalent strength when split non-mechanically (by hand) and mechanically (with a tablet splitter). In various embodiments, for any of the dividable tablets described herein, the dividable tablets optionally lose less than 3.0 percent mass between the divided subunits obtained when compared to the original whole tablet mass. In various embodiments, for any of the dividable tablets described herein, the divided subunits optionally meet finished product release requirements for dissolution.

The invention includes combinations of the aspects, embodiments and sub-embodiments of the invention disclosed herein.

### BRIEF DESCRIPTION OF THE FIGURES

The accompanying drawings illustrate the prior art and preferred embodiments of the invention and, together with a general description of the invention given above, and the detailed description given below, explain the principles of the invention.

FIG. 1 illustrates a perspective view of an incrementally layered tablet made from a three-dimensionally printed, bound powder matrix containing droxidopa.

FIG. 2 illustrates a cross-sectional view of the tablet of FIG. 1, taken along line 2-2 of FIG. 1.

FIG. 3 illustrates a printing pattern used to apply binding liquid to a base layer and top layer of build powder, for the tablet illustrated in FIG. 1.

FIG. 4 illustrates a printing pattern used to apply binding liquid to intermediate incremental layers of build powder, for the tablet illustrated in FIG. 1.

FIG. 5 illustrates a top perspective view of a three-dimensionally printed, bound-powder dividable tablet containing droxidopa, having a functional seam that extends along a dissecting line and joins or bonds together two sub-dosage units.

FIG. 6 illustrates the dividable tablet of FIG. 5 as a series of incremental, bonded layers of bound powder.

FIG. 7 illustrates a perspective view of the dividable tablet of FIG. 5 with an irregular segment removed from the sub-dosage units along the functional seam through line 7-7 of FIG. 5.

FIG. 8 illustrates a first printing pattern of the binding liquid used to print the top and bottom layers of the dividable tablet of FIG. 5.

FIG. 9 illustrates a second printing pattern of the binding liquid used to print the plurality of intermediate layers of the dividable tablet of FIG. 5.

FIG. 10 illustrates the dividable tablet of FIG. 5 that has been broken along the functional seam, dividing the tablet into two, typically equal, sub-dosage units.

FIG. 11 illustrates another embodiment of a dividable tablet having a physical score line formed within the top layer of bound powder.

FIG. 12 illustrates a printing pattern used in printing of the top layer of bound powder illustrated in FIG. 11.

### DETAILED DESCRIPTION OF THE INVENTION

### Definitions

As used herein “break strength” is a measure of an unreinforced tablet to resist failure in bending under a force applied along a lateral portion of the tablet, and typically a lateral centerline of the tablet.

As used herein, the term “derivative” refers to either a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps.

As used herein, the term “droxidopa” refers to the compound 2-amino-3-(3,4-dihydroxyphenyl)-3-hydroxypropanoic acid, and includes any and all stereoisomers, enantiomers, diastereomers, epimers, conformers, and/or racemates, whether isolated or unpurified, and salts thereof. Generally, droxidopa administered medicinally to patients is L-threo-dihydroxyphenyl serine ((2S,3R)-2-amino-3-(3,4-dihydroxyphenyl)-3-hydroxypropanoic acid). Accordingly, and unless otherwise indicated, 3DP dosage forms of the present invention that comprise droxidopa comprise L-threo-dihydroxyphenylserine. Droxidopa can also be synthesized or purchased as a salt, such as, in a non-limiting example, as a hydrochloride or methanesulfonate salt. The properties and synthesis of droxidopa are described in more detail in U.S. Pat. Nos. 3,920,728 and 11,447,443, the disclosures of which are incorporated by reference in their entireties.

As used herein, “ordinary handling conditions” means the typical conditions of manufacturing, processing, packaging, transportation, and storage to which tablet products of such kind are typically exposed in industry, including when handled by users.

As used herein, the term “pharmaceutically acceptable” refers to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

Throughout this description, the recitation of a numerical range with respect to any embodiment described herein also includes any value or sub-range between and inclusive of the larger range, as such values or sub-ranges are omitted for clarity. As a non-limiting example, an embodiment reciting a tablet comprising from 1 mg to 5,000 mg of droxidopa also recites, as non-limiting examples tablets comprising droxidopa at a mass of at least about 1, 25, 50, 100, 150, 200, 250, 300, 350, 400, 450, 500, 550, 600, 800, 1,000, 1,500, 2,000, or 2,500 mg, and/or up to about 5,000, 2,500, 2,000, 1,500, 1,000, 800, 600, 550, 500, 450, 400, 350, 300, 250, 200, 150, 100, 50, 25 mg, or about 100 mg, about 400 mg, about 600 mg, about 1,200 mg, about 100 mg to about 300 mg, or about 300 mg to about 600 mg.

Throughout this description, all masses and levels of components or compounds in a composition expressed as a ratio or a percentage are by weight, unless specified otherwise.

### Bound Powder Matrix

The droxidopa dosage forms described herein can be rapidly-orodispersible pharmaceutical compositions suitable for administration to a subject with a disease, condition, or ailment responsive to droxidopa. The dosage forms can be formed using additive manufacturing or three-dimensional-printing (3DP) techniques, particularly binder-jetting processes, to form a porous, bound powder matrix having a fixed three-dimensional shape. Methods for using binder-jetting processes to construct a bound powder matrix from a build powder and a binding liquid are described in further detail below. The bound powder matrices are rapidly orodispersible because they undergo immediate and very rapid disintegration when placed in a small volume of aqueous fluid, such as water, saliva, juice, milk, beverage, body fluid, soda, or a combination thereof.

The commercially-available droxidopa particles utilized in the droxidopa formulations described herein are generally sold in native form as a powder or particulate comprising, or consisting of, droxidopa compound. In some embodiments, the droxidopa particles can be processed to reduce and/or control their particle size, before being processed into the dosage forms. Non-limiting examples of methods to control droxidopa particle size are milling (optionally in the presence of hydrophobic silica), sieving, micronizing, grinding, or pulverizing.

The size, or equivalent diameter, of processed or unprocessed droxidopa, or any other particle, can be statistically expressed as a mean, a median, and/or size distribution within the sample. Typically, smaller particle sizes facilitate tighter packing of the particles within the dosage form's bound powder matrix, although particle sizes that are too small can cause difficulties with handling during the manufacturing process, including 3DP processes. Droxidopa particles used in forming the dosage forms described herein can have a volumetric mean particle size of, as non-limiting examples, greater than or equal to about 10, 25, 50, 75, 100, 125, 150, 175, 200, or 225 microns (μm); and/or less than or equal to 250, 225, 200, 175, 150, 125, 100, 75, 50, or 25 μm. Accordingly, and in some embodiments, the volumetric mean particle size of droxidopa contained within a dosage form of the present invention is about 10 to about 100 μm, or about 25 to about 50 μm.

Particles used in forming the dosage forms described herein can also be expressed as a percentage of particles, by volume, below a particular particle size. For instance, a particle size expressed as D(10) of ‘x’ microns indicates that 10% of the particles, by volume, within the particles sampled have an equivalent diameter of less than ‘x’ microns. Other common particle size values are D(50) (i.e., the value in which 50% of the particles, by volume, have an equivalent diameter less than the stated amount) and D(90) (i.e., the value in which 90% of the particles, by volume, have an equivalent diameter less than the stated amount).

In non-limiting examples, droxidopa particles within any of the dosage forms described herein can have: (a) a D(10) of at least about 1, 5, 10, 25, 50, 75, 100, or 125 μm, and/or up to about 150, 125, 100, 75, 50, 25, 10, or 5 μm, and about 1 to about 75 μm, or about 5 to about 50 μm; (b) a D(50) of at least about 5, 10, 25, 50, 100, 150, 200, 250, or 300 μm; and/or up to about 350, 300, 250, 200, 150, 100, 50, 25, or 10 μm, and about 10 to about 200 μm, or about 25 to about 100 μm; and (c) a D(90) of at least about 10, 25, 50, 100, 150, 200, 250, 300, 400, or 500 μm, and/or up to about 600, 500, 400, 300, 250, 200, 150, 100, 50, or 25 μm, and up to about 200 μm, or about 50 to about 150 μm.

Commercially-available droxidopa particles, and dosage forms manufactured from them, are often white or off-white upon their manufacture. However, droxidopa, as well as other monoamines and monoamine precursors, are susceptible to chemical aging, leading to the formation of pigments that cause the dosage form to change color, from white or off-white, to yellow, to brown, and/or to black. U.S. Pat. No. 8,980,316 describes the coloration of compressed tablets containing droxidopa, while the catechol-like compounds L-DOPA (3,4-dihydroxyphenylalanine) and carbidopa (N-amino-α-methyl-3-hydroxy-L-tyrosine monohydrate), the catecholamines dopamine and norepinephrine, and even aromatic amino acids as tyrosine and tryptophan, all of which are similar in structure to droxidopa, are all known to undergo oxidative and/or base-catalyzed degradation reactions to form melanin-like pigments (see, e.g., Vercruysse, K. P. and Whalen, M. M., “Light- or dark-colored, L-DOPA-based melanins”, Jun. 26, 2018; Asquith, R. S. and Rivett, D. E., “Photolysis of Tyrosine and its Possible Relationship to the Yellowing of Wool”, Textile Research Journal, July 1969, Volume 39, Issue 7, pg. 633-637). Accordingly, the present invention provides multiple dosage forms having components and formulations that delay or reduce the coloration under stressed environmental conditions, particularly elevated temperature and humidity conditions.

In particular, L-DOPA is a structural analog to droxidopa, differing from droxidopa by a single hydroxyl group. Without being limited by a particular theory, it is believed that the near structural identity between droxidopa, L-DOPA, and other monoamines and monoamine precursors, are likely to behave similarly under oxidative stress conditions.

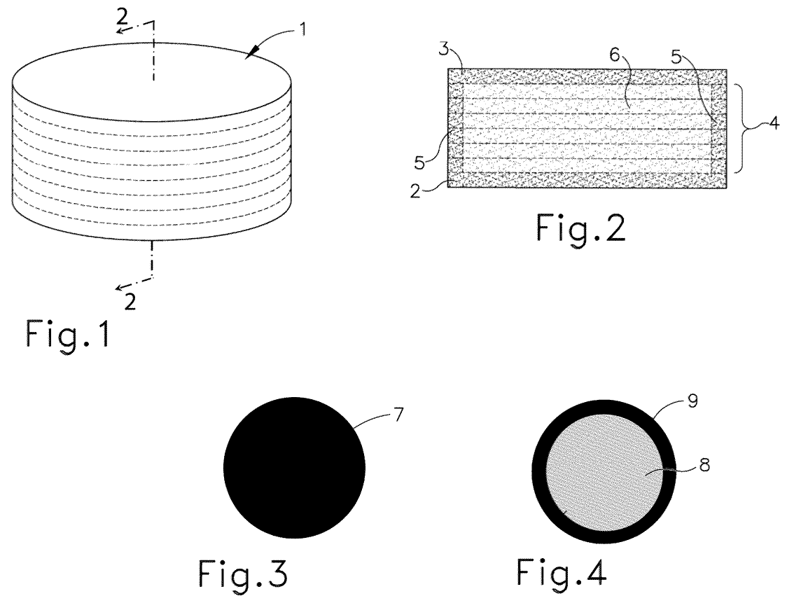

In a study looking at the rapid oxidation of L-DOPA in sodium hydroxide and monitored by Ultraviolet-Visible (UV-VIS) spectrophotometry, it was observed that the oxidation of L-DOPA led to the formation of a yellow solution having absorption bands at 330 nm and 440 nm (see, e.g., Madrakian, T., et al., “Simultaneous Derivative Spectrophotometric Determination of Levodopa and Carbidopa in Pharmaceutical Preparations” Bull. Korean Chem Soc. (2004) 25 (12): 1764-1768). The authors determined that the presence of the two absorption bands could be explained by the oxidation of L-DOPA (I), which has a λ_{abs,max} at about 280 nm, into an open-chain quinone (II), which has a λ_{abs,max} at 440 nm, and leucodopachrome (III), which has a λ_{abs,max} at 330 nm. The reaction scheme for the oxidation of L-DOPA to form the two chromophores is illustrated in FIG. 13A, and the UV-VIS absorption spectra for droxidopa and oxidized L-DOPA are illustrated in FIG. 13B (spectrum B) and FIG. 1C, respectively. In particular, FIG. 13C illustrates several overlain spectra of 10 mg L⁻¹ L-DOPA in the presence of IM NaOH at different times between 1-7 minutes, in which the up arrow indicates the passage of time and concomitant increase in the peak at 330 nm.

Another study was performed to examine the role of Fe²⁺ and hydrogen peroxide to cause oxidative stress form melanin-like pigments from L-DOPA (see Vercruysse, K. P. and Whalen, M. M., above). In one reaction condition in particular, the combination of Fe²⁺ and hydrogen peroxide together resulted in a solution that immediately turned light gold and ultimately to bright orange overnight, and slightly yellow solid pigment materials once dried. Other reaction conditions caused color changes ranging from light orange to black. The formation of colored complexes between L-DOPA and Fe³⁺ are also known via the compound's 3,4-dihydroxyphenyl moiety.

The coloration of droxidopa has been observed directly in preliminary studies examining the effect of temperature and humidity on the general stability of dosage forms stored in open blister containers. At 25° C. and up to 84% relative humidity (RH), various non-pH-controlled droxidopa three-dimensionally-printed (3DP) tablets of the present invention were shown to retain their native white color after 4.5 months. However, the tablets' resistance to color change decreased as a function of increased temperature, and particularly as a function of increased humidity. For example, tablets stored at 40° C. and 75% relative humidity (RH) had a noticeable color change to brown upon visual inspection at 4.5 months, whereas tablets stored at 40° C. but at RH values of 30% or lower had minimal to no color change. As the storage temperature increased even further, the relative humidity threshold decreased at which noticeable color change could be observed, as tablets stored at 30% RH and 50° C. turned a pale brown color.

The preliminary droxidopa stability studies described above, as well as studies directed toward determining the effect(s) of various components and processes on the coloration of droxidopa, are described in more detail in the Examples section, below. In particular, the addition of citric acid to a droxidopa formulation or dosage form was observed to reduce both the magnitude and rate of color change at elevated temperature and humidity (70° C./75% RH). Citric acid is a polyprotic organic acid having three acidic protons (pKₐ₁=3.13, pKₐ₂=4.76, and pKₐ₃=6.39), and is available commercially in either an anhydrous form or as a monohydrate. Without being limited by a particular theory, it is believed that other similar acidulant species, particularly those having at least two donatable protons with acidic pKₐ values, are likely to have a similar effect. Non-limiting examples of such acids are aspartic acid, citric acid, fumaric acid, glutamic acid, malic acid, maleic acid, malonic acid, oxalic acid, oxaloacetic acid, succinic acid, and tartaric acid. In non-limiting examples, a dosage form of the present invention can comprise an acidulant, particularly citric acid, at a percent by mass of at least about 0.1%, 0.3%, 0.5%, 0.7%, 0.9%, 1.0%, 1.25%, 1.5%, 1.75%, 2.0%, 2.5%, 3.0%, 3.5%, 4.0%, or 4.5%, and/or up to about 5.0%, 4.5%, 4.0%, 3.5%, 3.0%, 2.5%, 2.0%, 1.75%, 1.5%, 1.25%, 1.0%, 0.9%, 0.7%, 0.5%, or 0.3%, or from about 0.1% to about 0.5%, or from about 0.5% to about 1.0%.

When utilized in conjunction with a binder-jetting 3DP method, the acidulant can be provided via the binding liquid, via the build powder, or both. When acidulant is dissolved in the binding liquid, the acidulant becomes molecularly dispersed throughout the 3DP tablet, facilitating direct contact of the acidulant with the droxidopa particles within the bound matrix. When acidulant particles are included within the build powder, they remain as a plurality of particles distributed throughout the matrix of the 3DP tablet. The acidulant particles used in the build powder as a particulate can have D(90) of at least about 10, 25, 50, 100, 150, 200, 250, 300, 400, or 500 μm and/or up to about 600, 500, 400, 300, 250, 200, 150, 100, 50, or 25 μm, and for example a D(90) of citric acid particles of up to about 400, or about 200, or about 125, or about 100, or about 75 μm, and/or at least about 70, or about 100, or about 150 μm.

Generally, the 3DP bound powder matrices described herein comprise a particulate blend bound together by a binder. The particulate blend is provided via the build powder and comprises droxidopa and other particulate compounds. The binder comprises one or more binder materials, a portion of which can be present within the matrix as a particulate. The remainder of the binder can include one or more binder materials dissolved within a binding liquid, which is printed onto the build powder and forms bonds and bridges between the particulate material and dissolved portions of the build powder components. Collectively, the binder adheres together the particulates within the build powder, maintains the three-dimensional shape of the bound matrix prior to administering the tablet, and confers sufficient hardness and friability upon the tablet to maintain its structure under ordinary handling conditions.

In some embodiments, the binder is a water-soluble binder that can be included in the binding liquid and/or the build powder in binder-jetting processes. In some embodiments, the binding liquid can comprise water-soluble binder, by weight, at about 1% to about 20%, or about 5% to about 15%, or about 8% to about 12%. The build powder can comprise water-soluble binder, by weight, at about 1% to about 70%, or about 10% to about 55%, or about 20% to about 50%, and for example, about 5% to about 15%, or about 8% to about 14%, or about 9% to about 11%. The printed dosage form can comprise water-soluble binder, by weight, at about 5% to 80%, or at least about 10%, or 20%, or 30%, or 40%, or 50%, or 60%, or 70%, and/or up to 75%, or 65%, or 55%, or 45%, or 35%, or 25%, or 15%, and for example, about 40% to about 60%, about 35% to about 55%, or about 45% to about 60%. Non-limiting examples of binder materials that can be present within any of the dosage forms described herein are mannitol, polyvinylpyrrolidone (also interchangeably known as “povidone”), poly(1-vinylpyrrolidone-co-vinyl acetate) (also interchangeably known as “copovidone”), lactose, fructose, sucrose, dextrose, sorbitol, xylitol, lactitol, erythritol, pregelatinized starch, modified starch, hydroxypropyl cellulose, and hydroxypropyl methylcellulose.

When binder is included as a particulate material in the build powder, an excess of binder beyond the amount required to form a stable bound matrix can be present within the dosage form as a filler material. In some embodiments, the filler material can comprise one or more of the above sugars or sugar alcohols on account of their low-strength binding and inertness relative to droxidopa. In some embodiments, the filler material comprises, or alternately, consists of, mannitol. In non-limiting examples, any dosage form of the present invention can comprise mannitol at a percent by mass of at least about 1%, 2.5%, 5%, 7.5%, 10%, 15%, 20%, 25%, 30%, 35%, 40%, 45%, 50%, 55%, 60%, 65%, or 70%, and/or up to about 75%, 70%, 65%, 60%, 55%, 50%, 45%, 40%, 35%, 30%, 25%, 20%, 15%, 10%, 7.5%, 5%, or 2.5%.

The dosage forms of the present invention are rapidly orodispersible, and can disperse (disintegrate) in the mouth, with or without a sip of liquid, or in a volume of aqueous liquid, within about 30 seconds, or within about 20 seconds, or within about 15 seconds, or within about 10 seconds, or within about 3 seconds, or within about 1 second. The aqueous liquid can be saliva, gastric fluid and/or water, in a volume of 50 ml, or less, or 20 ml or less, or 15 ml or less, or 5 ml or less, or 1 ml or less, even when the mass of droxidopa is greater than 300 mg. Without being limited by a particular theory, it is believed that these dosage forms can disintegrate quickly because their bound matrix is porous, facilitating quicker contact of the solvent with the inner core of the 3DP tablet. Disintegration time can also be determined according to the procedure described in the United States Pharmacopeia (USP)<701>. When tested for disintegration according to USP <701>, the dosage forms of the present invention can disintegrate within about 30 seconds, or within about 20 seconds, or within about 15 seconds, or within about 10 seconds, or within about 3 seconds, or within about 1 second.

To facilitate rapid disintegration of the matrix within the patient's oral cavity, any of the dosage forms described herein can also comprise a hydrophilic disintegrant that can assist in separating the bound powder matrix into smaller particles upon contacting moisture, wherein the disintegrant comprises one or more disintegrant compounds. Non-limiting examples of disintegrant compounds are microcrystalline cellulose, cross-linked polyvinylpyrrolidone (also interchangeably known as “crospovidone”), croscarmellose, and sodium starch glycolate. In some embodiments, the disintegrant comprises microcrystalline cellulose. In some embodiments, the disintegrant consists of microcrystalline cellulose. In non-limiting examples, any dosage form of the present invention can comprise a disintegrant at a percent by mass of at least about 1%, 3%, 5%, 7.5%, 10%, 15%, 20%, 25%, or 30%; and/or up to about 35%, 30%, 25%, 20%, 15%, 10%, 7.5%, 5%, or 3%. Generally, disintegrant compounds can be processed into the tablet as particulates blended uniformly into the build powder, to minimize contact with moisture prior to manufacturing the dosage form.

Any of the dosage forms described herein can optionally further comprise glycerin, which has been reported to increase the hardness of 3DP dosage forms while reducing disintegration time, and it can also be used as a humectant, sweetener, preservative, lubricant, saponifier, viscosity modifier, or solvent. In a binder jetting process, glycerin can be used as a dissolved component within the binding liquid used to inhibit blockages and stoppages of the printing nozzles, as described in U.S. Pat. Nos. 9,339,489, 10,420,785, and 9,616,018, the disclosures of which are incorporated by reference in their entireties. Glycerin can also be provided via the build powder in salt form, such as glycerin monostearate. In non-limiting examples, any dosage form of the present invention can comprise glycerin at a mass percent of at least about 0.1%, 0.25%, 0.5%, 0.75%, 1.0%, 1.25%, 1.5%, 2.0%, 3.0%, 4.0%, 5.0%, or 7.5%, and/or up to about 10%, 7.5%, 5.0%, 4.0%, 3.0%, 2.0%, 1.5%, 1.25%, 1.0%, 0.75%, 0.5%, or 0.25%, or from about 0.1% to about 1.0%, or from about 0.5% to about 0.75%.

However, as described in the examples below, initial formulations of binder-jetted dosage forms comprising droxidopa and glycerin developed color at an apparently accelerated rate relative to droxidopa dosage forms prepared without glycerin. Without being bound by any particular theory, it is believed that compositions comprising droxidopa are less stable in the presence of glycerin because glycerin can act as a humectant and retain moisture within the dosage form. Accordingly, the mass of acidulant added to the formulation can be controlled to retain the performance benefits gained by using glycerin in the first place, while simultaneously delaying, reducing, and/or inhibiting the enhancement in coloration attributable to glycerin. Non-limiting examples of mass ratios of acidulant to glycerin are about 1:1, about 1:2, about 1:3, about 1:4, about 1:5, about 1:6, about 2:1, about 3:1, about 4:1, about 5:1, or about 6:1, and for example, from 1:2 to 1:7, or from 1:3 to 1:6.

Generally, a printed dosage form can be expressed by the dosage form's total mass of droxidopa. As non-limiting examples, any of the dosage forms described herein can comprise droxidopa at a mass of at least about 1, 25, 50, 100, 150, 200, 250, 300, 350, 400, 450, 500, 550, 600, 800, 1,000, 1,500, 2,000, or 2,500 mg; and/or up to about 5,000, 2,500, 2,000, 1,500, 1,000, 800, 600, 550, 500, 450, 400, 350, 300, 250, 200, 150, 100, 50, or 25 mg, or about 100 mg to about 600 mg, or about 300 mg to about 1,200 mg, or about 300 mg to about 600 mg, or about 400 mg to about 600 mg, or about 200 mg to about 1,200 mg, or about 400 mg to about 800 mg. Additionally, any of the dosage forms described herein can be expressed as a percent by mass of droxidopa within the dosage form. As non-limiting examples, any of the dosage forms herein can comprise droxidopa at a percent by mass of at least about 1%, 5%, 10%, 15%, 20%, 25%, 30%, 33%, 35%, 40%, 45%, 50%, 55%, 60%, 65%, 66%, 70%, or 75%; and/or up to about 80%, 75%, 70%, 66%, 65%, 60%, 55%, 50%, 45%, 40%, 35%, 30%, 25%, 20%, 15%, 10%, or 5%.

In some embodiments, the bound powder matrix comprises, by weight: (a) droxidopa at up to 80%, preferably up to 66%, more preferably 20% to 50%, and even more preferably 30% to 35%; (b) disintegrant at 3.0% to 35%, preferably 5% to 20%, and more preferably 10% to 15%; (c) binder, preferably at 20% to 75%, more preferably 45% to 65%, and (d) acidulant at up to 10%, preferably up to 5%, more preferably up to 2%, and even more preferably up to 1%. In some embodiments, the bound powder matrix further comprises: (e) glycerin at up to 5%, preferably up to 2%, and more preferably up to 1%. In some embodiments, the bound powder matrix further comprises, by weight: (f) a glidant, for example colloidal silicon dioxide, at up to 2%, preferably up to 1%, and more preferably up to 0.5%. In some embodiments, the bound matrix further comprises, by weight: (g) a surfactant, for example polysorbate 20, polysorbate 40, polysorbate 60, polysorbate 80, sodium lauryl sulfate, poloxamer 124, poloxamer 188, poloxamer 237, poloxamer 338, poloxamer 407, and PEG 400, at up to 4%, preferably up to 2%, and more preferably up to 1%. In some embodiments, the bound matrix further comprises one or more of a preservative, a chelating agent, an antioxidant such as ascorbic acid, butylated hydroxyanisole (BHA), butylated hydroxytoluene (BHT), n-propyl gallate, L-cysteine HCl, sodium bisulfite, sodium sulfite, Vitamin E, methionine, sodium metabisulfite, and EDTA, a filler, a lubricant, a solvent, a saponifier, and a diluent.

### Dosage Form Manufacturing

Generally, dosage forms and their formulations of the present invention can be manufactured using any known method in the art, including binder jetting processes.

In various embodiments, 3DP droxidopa dosage forms of the present invention can be built using any conventional additive manufacturing or 3DP freeform fabrication system and/or equipment assembly that utilizes a build platform configured to build objects from a bed or other supply of powder material, non-limiting examples include those described in U.S. Pat. Nos. 6,471,992, 8,888,480, 9,339,489, 9,492,380, 9,616,018, 11,383,440, and 11,433,022, the disclosures of which are incorporated by reference in their entireties. When an API comprises a powder or other solid particles, the additive manufacturing of one or more dosage forms using 3DP can utilize the steps of: depositing a pre-determined quantity of a build powder onto a height-adjustable build surface; spreading the build powder into a thin layer having a substantially uniform thickness; and dispensing a binding liquid onto the thin powder layer to bind together the particles of build powder in a defined pattern within the thin powder layer. The successive steps of depositing a thin layer of build powder and dispensing binding liquid are repeated, usually several times, until the printing of the one or more dosage forms is complete. Each completed dosage form can be separated from the unbound powder (when present), and excess solvent or water from the dispensed binding liquid can be evaporated out of the completed dosage form(s) to generate the final tablet(s) comprising a porous bound powder matrix.

In various embodiments, the droxidopa dosage forms of the present invention can be built using additive manufacturing or 3DP manufacturing systems and/or equipment assemblies configured for forming objects in situ within the depression of a packaging material, particularly any of the systems or assemblies described in U.S. Pat. No. 11,278,501 and International Patent Publication WO 2021/211898, the disclosures of which are incorporated by reference in their entireties. The packaging can comprise one or more depressions, and in some embodiments, a pattern of a plurality of depressions. Non-limiting examples of such packaging are a blister pack and a disposable single-dose blister pack.

In various embodiments, which may be used in combination with any other embodiment described herein, a dosage form containing droxidopa can be formed into any three-dimensional geometric shape. In some embodiments, the dosage form can be in the shape of an irregular or regular polyhedron, a prism having an “n” number of regular faces, for example, 3, 4, 5, 6, or 8 regular faces, a prismatoid, a prismoid, a scutoid, a frustum, such as a pyramidal frustum, a conical frustum, a spherical frustum, and frustoconical sections. Non-limiting examples of dosage form shapes can include square, circular, and elliptical cylinders that can conveniently be bisected by a functional seam. In other embodiments, a rapidly-orodispersible dosage form can have one or more rounded surfaces, such as, as non-limiting examples, top and/or bottom rounded surfaces, including spherical, ellipsoidal, and/or spherocylindrical (capsular) shaped surfaces. Particularly, rapidly-orodispersible dosage forms formed with rounded surfaces, particularly spherocylindrical dosage forms, can be formed to mimic the capsular and ellipsoidal shapes of other commonly prescribed and over-the-counter medications.

In some embodiments, the rapidly-orodispersible dosage form may comprise a wide flat cylindrical shape having rounded edges, optionally having one or more surface depressions to aid subdivision of the dosage form into subunits, and/or optionally having one or more surface depressions to serve as identifying marks for the intact dosage form and/or the subunits of the dosage form. Those skilled in the art would appreciate that the above examples are non-limiting, and that there are an infinite number of shapes into which each dosage form can be constructed.

3DP dosage forms having a bound matrix comprising droxidopa are generally prepared according to the procedures described below in Example 1, Example 6, and Example 7. The remaining examples describe the evaluation of the chemical and physical properties of the resulting dosage forms to identify components, procedures, and environmental conditions that can provide a balance of improved chemical stability, sufficient hardness, low friability and extremely rapid disintegration time in a small volume of aqueous liquid.

Generally, 3DP dosage forms of the present invention have from at least 3 layers and up to at least 50 layers, or from at least 10 layers up to 50 layers, or from at least 15 layers up to at least 45 layers, or from at least 20 layers up to at least 40 layers, or from at least 5 layers up to at least 15 layers, or from at least 5 layers up to at least 10 layers of bound powder within their bound matrices. In some embodiments, the 3DP dosage form has at least five layers of bound powder comprising its bound matrix.

Further, the substantially-uniform thickness (vertical height) of each of the layers can be controlled. In some embodiments, all the layers within a 3DP dosage form can have the same substantially-uniform thickness. In some embodiments, the substantially-uniform thickness within one or more layers within the 3DP dosage form can be varied relative to other layers within the same dosage form. In some embodiments, a layer within a 3DP dosage form can have a substantially-uniform thickness of at least about, 25, 50, 100, 150, 200, 250, 300, 350, 400, or 450 microns, and/or up to about 500, 450, 400, 350, 300, 250, 200, 150, 100, or 50 microns, or about 100 microns to about 400 microns, or about 300 microns to about 500 microns, or about 150 microns to about 300 microns. In some embodiments, each of the layers within a 3DP dosage are about 400 microns thick.

In some embodiments, a finished 3DP dosage form, prior to packaging, can comprise not more than 5%, or 3%, or 2%, or 1.5%, or 1% of moisture as determined by loss at a drying temperature. In some embodiments, the drying temperature for drying a 3DP dosage form containing droxidopa particles is from 100° C. to 120° C.; for example, about 105° C.

In some embodiments, a 3DP dosage form can have a hardness that is about the same, and in some embodiments, uniform, in any orientation of the tablet, as determined by a tablet breaking force assay according to USP <1217>, of from about 1 kilopond (kp) to about 20 kp, or about 1 kp to 10 kp, about 1 kp to about 7 kp, about 1 kp to about 5 kp, about 3 to about 9 kp, or about 3 to about 6 kp.

In some embodiments, the hardness of portions of the bound matrix can be controlled such that localized portion(s) of the matrix are denser (or less porous) and/or harder than others. In some embodiments, the hardness of an exterior surface portion of the bound matrix is greater than the hardness of an interior portion of the bound matrix. The hardness of a portion of a bound matrix can be attained and controlled by the localized quantity of binding liquid, and consequently, the respective quantity of dissolved binder applied to such portion, wherein greater proportions of binder result in increased hardness.

### Binder Jet Printing

Three-dimensional printing of binding liquids can have spatial descriptors in each of three different, typically orthogonal directions. In three-dimensional printing, binding liquid may be dispensed from a single print port of a print nozzle in droplets or in liquid units resembling droplets. A layer of powder material can be moved in a horizontal plane directly beneath a print port, in a longitudinal direction of motion. The single print port can be moved laterally relative to a surface of the layer of the powder material while dispensing droplets vertically from the single print port in a succession that deposit onto the surface of the layer of powder material, also referred to herein as a layer of build powder, to form a line of droplets corresponding to the line of movement of the printhead over the layer of build powder. The spacing between the successive droplets deposited in the lateral line on the surface of the build powder layer is referred to as a drop-to-drop spacing, or droplet spacing. After completion of one lateral line of droplets along the surface of the layer of build powder, the layer of build powder is repositioned in a longitudinal direction relative to the printhead, and another lateral line of droplets is deposited adjacent to the previously-deposited line of droplets, and separated longitudinally therefrom by a distance referred to as a line-to-line spacing. After completion of printing on a layer of powder, another powder layer may be deposited, with each powder layer having a layer thickness. The powder layer thickness is the third descriptor.

In the printing of binding liquid onto a layer of build powder, the spacing of droplets from a single print port deposited along the moving surface of the layer of build powder moving below the print nozzle may be described in terms of the resolution of the printing system, often expressed as dots per inch (dpi), where a dot is a single droplet and dpi is the reciprocal of droplet spacing. For example, resolutions of 300 and 600 dpi correspond to droplet spacings of about 84.7 microns and about 42.3 microns, respectively. The droplet spacing (within a line), or the line spacing (spacing of droplets from one line to the next), or any other spacing of droplets may be described in terms of resolution expressed in dpi. One can determine the number of droplets applied to a specified area of the top surface of a powder layer by the droplet spacing and the line spacing, and conversely, given the required number of droplets required on a specific area, a droplet spacing and line spacing can be determined.

In various embodiments, the print nozzle comprises a series of print ports in a row or linear series. Typically, the series of print ports will be arranged laterally, transverse to the longitudinal direction of relative movement of the layer of build powder in the horizontal plane. Generally, a layer of powder will move horizontally in the longitudinal (machine) direction under an array of print ports. The binding liquid dispensed from each port of the lateral series of print ports forms a longitudinal line of successive droplet deposits onto the layer of build powder moving beneath the print ports. The frequency of droplets dispensed from a print port is described as droplets per second (hertz, or Hz), and depending on the velocity of the layer of build powder moving longitudinally beneath the print port, the deposits of printing liquid along the longitudinal line are described as droplets per unit length, such as droplets per inch or droplets per centimeter.

The distance between adjacent print ports defines the lateral distance between the longitudinal lines of successive droplet deposits, which is defined as the line-to-line spacing, or line spacing, which is the reciprocal of the number of print ports per unit of length, such as per inch or per centimeter.

Another droplet factor is the volume or corresponding diameter, and the corresponding mass, of the droplets. Controlling the size, and the corresponding mass, of the droplets for a selected droplet resolution allows control of the total mass of binding liquid applied to the specified area of the top surface of a powder layer.

In the printing of binding liquid onto layers of build powder to form one or more printed article, a print pattern is generated for each incremental layer of build powder that forms the printed article. The print pattern instructs the print nozzle on the timing, size (mass quantity) and frequency of the droplets to be dispensed from selected ports of the print nozzle to achieve both the shape of the print pattern of droplets and the mass or volume of the binding liquid deposited onto each areal portion of the layer of build powder corresponding to the print pattern.

In the printing of binding liquid onto a single layer of build powder, the print pattern can be represented by a pixelated image characterized by a resolution in dots-per-length (for example, dots-per-inch) in each of two orthogonal linear directions, each dot corresponding to a droplet. In some instances, these pixelated images are 1-bit monochrome images, alternately referred to as binary or bi-level images in which each pixel contains one bit of information (0 or 1) that may be represented as either black or white onscreen. A pattern in which all pixels are black is referred to as a “solid” printing pattern. For making dosage forms from a series of layers of build powder, a series of pixelated images is prepared and used, representing the layer-by-layer printing instructions for each succussive layer of build powder for the printing of the article.

In some instances, an amount of binding liquid applied in a localized areal region(s) of the dosage form can be achieved by using a combination of solid printing and “grayscale” printing, which uses a grayscale print pattern in the dosage form design. In the case of 1-bit monochrome images used for machine instructions, grayscale printing is achieved by changing the number of “black” pixels relative to “white” pixels in a chosen region of a dosage form, or in a chosen layer of a dosage form, or throughout a dosage form. Other regions may be “solid” by using all black pixels. In some embodiments, the dosage form design includes a “solid” printed exterior or periphery, and a “grayscale” printed interior, which results in fewer droplets of liquid per unit area within the interior of the printed region as compared to the area of the outer periphery. In some embodiments, grayscale printing may be achieved with equally spaced black pixels amongst white pixels to reach an overall ratio of black to white pixels in the grayscale printed region. In other embodiments, grayscale printing may be achieved with randomly placed black pixels amongst white pixels to achieve an overall ratio of black to white pixels in the grayscale printed region. In still other embodiments, grayscale printing may be achieved with a chosen pattern (e.g., parallel lines, hashed pattern, dot pattern) of black pixels amongst white pixels to achieve an overall ratio of black to white pixels in the grayscale printed region.

In some embodiments for the printing of binding liquid onto a layer of build powder to form one or more printed article, the saturation level of binding liquid applied to a selected surface area(s) or region(s) of a powder layer surface can be expressed as an applied quantity of the binding liquid within a selected area of the powder layer to be wetted. To account for the depth of a powder layer, and thus the volume of powder being wetted by a quantity of the binding liquid, the saturation level can be defined as the volume or mass, for example milligrams (mg), of binding liquid applied per unit of surface area of the powder layer, for example square centimeters (cm²), per thickness of the powder layer, and typically the average thickness of the powder layer, for example millimeters (mm).

In various embodiments, a saturation level of a binding liquid onto a layer of powder is about 1 to 60 mg/cm²/mm, for example, about 4 to 32 mg/cm²/mm. The specific saturation of a binding liquid will vary depending, without limitation, upon the components of the binding liquid, the components of the powder material that forms the powder layer, the size distribution of the particles of the powder material, and the desired properties of the bound powder matrix of the printed tablet.

A full saturation level of binding liquid is a quantity, including either a mass quantity or a volumetric quantity, of binding liquid applied as droplets to a unit area of a layer of powder material per unit depth of the layer, that is sufficient to migrate downwardly through the powder material within the unit area to a full depth of the layer of powder material, and to wet the powder material within the print area sufficiently to bond the particles of the powder material together into a unitary bound powder matrix that has a tenacity sufficient to avoid breaking thereof under ordinary handling conditions, though without overly or excessively saturating and dissolving most or all of the water-soluble components of the powder material. The quantity of binding liquid deposited onto the layer of powder material will typically migrate into the powder material surrounding the area where the liquid was deposited, and dissolve some amount of any dissolvable compound within the powder material that is contacted. Excessively low saturation levels tend to result in poor structural integrity of the resulting bound powder material. Exceedingly high saturation levels tend to result in excessive bleeding of liquid beyond where the liquid was deposited and intended to flow, and potentially excessive dissolving of compounds of the powder material into the binding liquid. A person of ordinary skill in the art, for a particular powder material and binding liquid, will be able to determine and define a full saturation value of a binding liquid with minimal testing.

A full or 100% saturation level hereinafter can then be determined and designated for a particular powder material and binding liquid. In some embodiments, a full or 100% saturation level is about 8 to 60 mg/cm²/mm of binding liquid, including 10 to 40 mg/cm²/mm, and more typically about 12 to 32 mg/cm²/mm. Generally, use of more than full or 100% binder saturation is not needed to provide effective bonding of the powder material, and can unnecessarily increase the drying requirements to evaporate excess free binding liquid.

Having determined a 100% saturation, a binding liquid can be applied onto a particular area of the powder material layer in a quantity of 100% saturation, or more, or less than 100% saturation. For example, a binding liquid can be applied onto a particular area of the powder material layer in a quantity less than 100% saturation, such as to 90%, or 80%, or 70%, or 60%, or 50%, or 40%, or 30%, or 20%, or 10% saturation, or saturations therebetween. In general, saturation levels less than full or 100% saturation for a particular powder material and binding liquid are also referred herein as “grayscale” saturations.

In various embodiments, the quantity of binding liquid within a selected area of a powder layer is about 0.6 mg/cm²/mm to about 60 mg/cm²/mm, and more preferably about 6.2 mg/cm²/mm to about 40.0 mg/cm²/mm. In various embodiments, a level of binding liquid to be applied to at least the peripheral portions (sidewalls, and top and bottom surfaces) of the completed tablet is typically about 25.0 to about 35.0 mg/cm²/mm, and for example, about 30.0 to about 32.0 mg/cm²/mm, and is sufficient to provide structural integrity to the finished tablet during handling.

Suitable printing devices include those having a continuous inkjet printer (CIJ) or those having a drop-on-demand printhead. A continuous jet printhead provides a continuous jet (spray) of droplets that can be selectively charged while passing through plates and targeted onto a powder layer, while uncharged droplets are not deflected and return to the CIJ system. A drop-on-demand printhead only deposits droplets of printing fluid onto the powder layer if it receives an instruction (demand, operational command) to do so. A printhead scans (applies fluid to) the surface of a powder layer at a predetermined rate, e.g. a scan rate, to form a line of droplets. A high scan rate will result in a lower saturation level, and a low scan rate will result in a higher saturation level when comparing printing fluid deposition at a constant volume per unit time. An increase in the scan rate from 1.0 m/s to 2.0 m/s reduces the total volume of binder solution deposited in the tablets by half. As the print speed increases, the apparent density of the printed article (theoretical, calculated from the weight and dimensions of the tablet) decreases. A simultaneous decrease in the dimensions and weight of the tablets is also seen. This decrease is attributed to the fact that a decrease in the total volume of binder droplets deposited onto the powder results in a decrease in the extent of binder solution spreading in the powder. Increasing the print speed also decreases the flash time and the hardness and increases the friability of the tablets. This result is obtained because the proportion of binding liquid decreases in the tablets as the print speed increases. An increase in the print speed also increases the void volume inside the tablets, as illustrated by an increase in the percent volume of the tablets penetrated by mercury at 30 psi (% intrusion).

When using a continuous jet printhead, the printhead scans at a rate of about 0.5 to 3.0 m/sec, and most preferably at about 1.75 m/sec. When using a drop-on-demand jet printhead, the printhead scans at a rate of 0.1 to 1 m/sec, most preferably at about 0.15 to about 0.5 m/sec.

The volume of individual droplets can be varied as desired, for example, by selection of a different three-dimensional printing machine, or different printhead components on the same machine, or different parameters on the same printhead and same machine. Increasing the volume of the droplet increases the saturation level and decreasing the volume of a droplet decreases the saturation level when comparing printing fluid deposition at a constant scan rate. When using a continuous jet printhead, the size of the fluid droplets delivered by the printhead preferably ranges from about 15 μm to about 150 μm in diameter. When using a drop-on-demand printhead, the size of the fluid droplets delivered by the printhead preferably ranges from about 40 μm to about 60 μm in diameter.

The flow rate of the liquid delivered by the printhead can be varied as desired. Increasing the flow rate will increase the saturation level by increasing the droplet size and/or droplet resolution. Decreasing the flow rate decreases the saturation level when comparing printing fluid deposition at a constant scan rate. As discussed herein, the printhead deposits droplets of printing fluid to form parallel lines thereof in the powder layer. When using a continuous jet printhead, the line spacing ranges from about 20 to about 1000 μm, or about 50 to about 500 μm, and preferably about 100 to 200 μm. When using a drop-on-demand jet printhead, the line spacing ranges from about 20 to about 300 μm, about 40 to about 100 μm, or about 55 to 75 μm.

The instructions for printing of the print pattern take into account parameters of the build powder and the binding liquid, and the movement velocity of the layer of build powder relative to the printhead and its ports. The parameters of the build powder can include the mass portion and hydroscopicity of the various build powder components. In general, water-absorbing or water-soluble powder materials will take up more binding liquid than a water-repelling or water-insoluble material, while requiring less binding liquid to form a wetted bound powder matrix that, once dried by removing excess water and solvent, forms a stable, hard and non-friable bound powder matrix. The parameters of the binding liquid can include the mass portion of water and of solvents, and the mass portion of any dissolved binder components used in the binding liquid.

### Dividable Tablets

If a conventional tablet 1 such as one shown in FIG. 1 is struck with a utensil along the centerline of the top of the tablet, the conventional tablet possibly would break into two or more pieces, and the interior portions 6 of the intermediate layers 4 would be exposed. It would be possible that the tablet will not break along the centerline into two equal or functionally equivalent parts. It is also possible that the bound powder matrix in the interior portions 4 (FIG. 2) may fall away from the bound powder matrix of the interior portions 4, resulting in a loss of the powder material within the interior portions 4. This would be problematic for a medicament or pharmaceutical tablet or dosage form, especially if droxidopa were contained within the ingredient powder of the bound powder matrix.

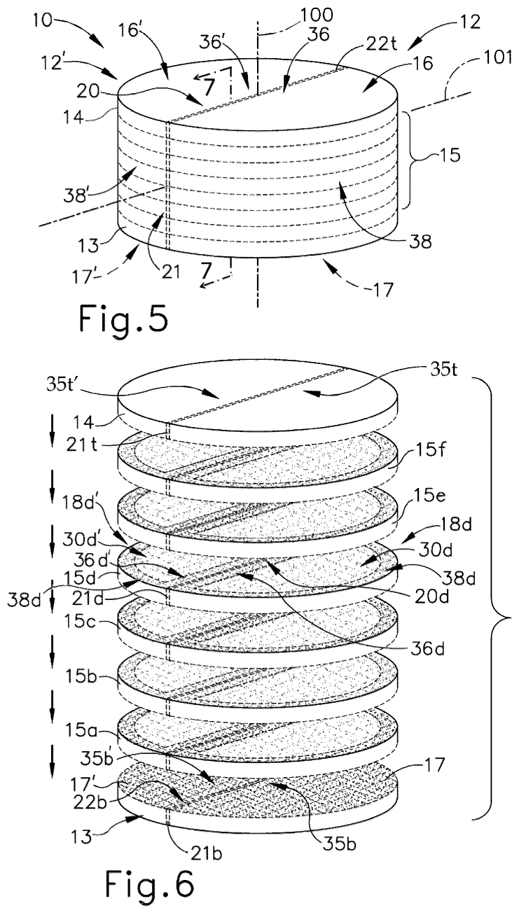

A dividable tablet 10 is illustrated in FIGS. 5-7. FIG. 5 illustrates a top perspective view of the dividable tablet 10 having a functional seam 20 comprising, and typically consisting of, a narrow planar portion of a bound powder matrix that extends along a bisecting line 101 and joins or bonds together two opposing planar, breakage-resistant boundary walls 36, 36′ (FIG. 5) of the two sub-dosage units 12,12′ (see also boundary wall portions 36d,36d′ in FIG. 6). The bound powder matrix of the functional seam 20 has a lower break strength than the adjoining boundary walls, to provide improved subdivision of the tablet into the two (or more) sub-dosage units 12,12′. Each of the two (or more) sub-dosage units 12,12′ includes a body that includes respectively the planar breakage-resistant boundary wall 36,36′, an outer peripheral wall 38,38′, the top wall 16,16′, and the base wall 17,17′. The functional seam 20 extends axially to bisect both a base bound-powder layer 13 and a top bound-powder layer 14 and extends laterally to sidewall portions 21 that bisect the outer peripheral wall 38,38′ of the sub-dosage units 12,12′ of the tablet 10.

The dividable tablet 10 comprises, and typically consists of, a plurality of bound-powder layers, including the one or more base bound-powder layer 13, the one or more top bound-powder layer 14, and a plurality of intermediate bound-powder layers 15, which in the illustrated embodiment of FIG. 5 includes six intermediate bound-powder layers 15a through 15f. FIG. 6 shows the dividable tablet 10 as a series of incremental, bonded layers of bound powder.

The one or more base bound-powder layer 13 (shown in FIG. 6) comprises a base functional seam portion 22b that extends to the peripheries 21b to join or bond together the pair of opposed base wall portions 17,17′ including the base boundary wall portion 35b,35b′ that join or bond along opposite sides of the base functional seam portion 22b.

The top bound-powder layer 14 (shown in FIG. 5) comprises a top functional seam portion 22t that extends to the sidewall portion 21t to join or bond together the pair of opposed top wall portions 16,16′ including top boundary wall portion 35t,35t′ joined or bonded along opposite sides of the top functional seam portion 22t.

Each of the plurality of intermediate bound-powder layers 15 (referred to collectively as layers 15x, and individually as layers 15a-15f) comprises respectively an intermediate functional seam layer portion 20 (referred to collectively as portions 20x, and individually as portions 20a-20f), and opposed intermediate sub-layer portions 18 and 18′ (referred to collectively as layer portions 18x, and individually as layer portions 18a-18f) joined or bonded along opposite sides of the intermediate functional seam layer portions 20x.

The top of the fourth intermediate layer 15d shows the intermediate functional seam layer portion 20d joined to and separating two half-layers identified as the intermediate sub-layer portions 18d, 18d′. Each intermediate sub-layer portion 18d, 18d′ consists of a boundary wall portion 36d,36d′, a peripheral wall portion 38d,38d′, and an intermediate interior portion 30d,30d′. The other five intermediate layers 15a-15c and 15e-15f each have the same construction as the fourth intermediate layer 15d, though can be different.

The functional seam 20 of the dividable tablet 10 therefore consists of a series of incremental bound-powder layers that include the base functional seam portion (22b, FIG. 6), the plurality of intermediate functional seam portions (20a-20f, FIG. 6), and the top functional seam portion 22t (FIG. 5), stacked vertically and bound to one another. Likewise, the boundary walls 36,36′ consist respectively of the base boundary wall portions 35b,35b′ of the base layer 13, the intermediate boundary wall portions 36x,36x′, and the top boundary wall portion 35t,35t′, stacked vertically and bound to one another.

The sub-dosage unit 12 (and the opposed sub-dosage unit 12′ respectively) shown in FIG. 5 consists of the planar, breakage-resistant boundary wall 36, hereinafter referred to just as “boundary wall”, the peripheral wall 38, base wall 17, top wall 16, and interior portions 30x.

The boundary wall 36 consists of a series of layers that include the base boundary wall portion 35b of the base layer 13 shown in FIG. 6, the intermediate boundary wall portions 36a-36f of the intermediate layers 15x, such as intermediate boundary wall portion 36d of intermediate sub layer 15d shown in FIG. 6, and the top boundary wall portion 35t of the top layer 14 shown in FIG. 5, which are joined together during the 3D printing process.

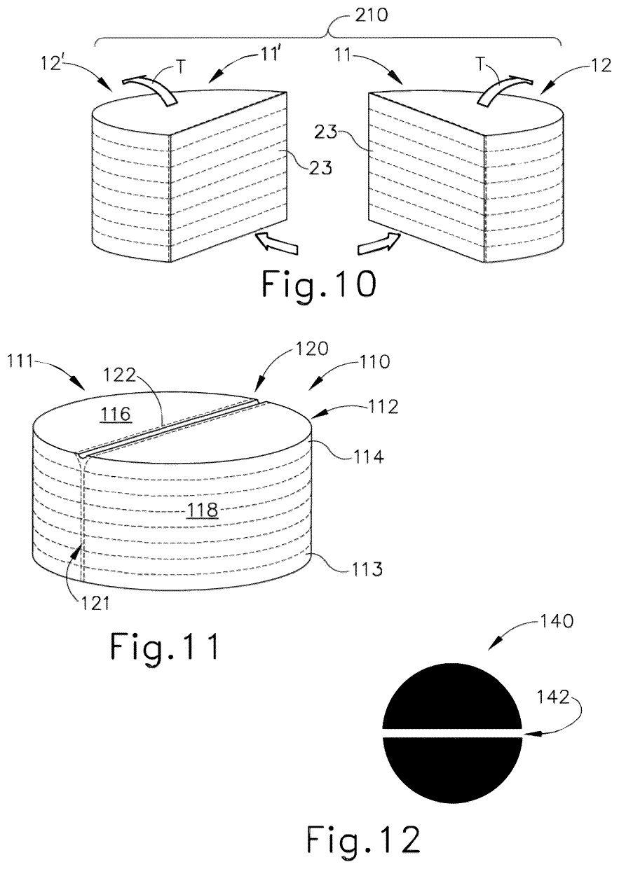

The peripheral wall 38 consists of the peripheral wall portions 38x of the intermediate bound-powder layers 15x (see FIG. 6). The peripheral wall 38, the top wall 16, and the base wall 17, with the respective boundary wall 36, form a unitary outer shell of each sub-dosage unit 12. The peripheral wall 38, the top wall 16, and the base wall 17 components of the outer shell independently comprise a third bound powder matrix that has a third break strength, which is typically greater than the break strength of the bound powder matrix of the functional seam 20, to ensure that both the boundary walls 36,36′ and the sub-dosages units 12,12′ do not crack or break when the tablet 10 is divided along the functional seam 20 into the two separate sub-dosage halves 11,12 (see FIG. 10).

The intermediate interior portions 30x,30x′ of the tablet 10 can comprise a bound powder matrix or an unbound powder. In various embodiments, the intermediate interior portion 30x,30x′ of the tablet 10 comprise a bound powder matrix having a break strength that neither contributes to nor diminishes the breaking of the tablet into sub-dosage units. As an increase in a break strength of a portion of the bound powder matrix can increase with binder content, and decrease possibly the orodispersive quality and/or disintegration rate of such portion of the sub-dosage units, it can be preferred to minimize the binder content, and therefore the break strength, of the intermediate interior portions 30x,30x′ of the tablet 10, to ensure target disintegration rate while minimizing and avoiding breakage of the sub-dosage units when dividing the tablet along the functional seam 20.

Preferably the two or more sub-dosage units 12,12′ have equal mass and ingredient content after dividing. Forming of the functional seam 20 between the boundary walls 36, 36′, as shown in FIGS. 5 and 6, can be done using a similar process as described above for forming the conventional dosage forms shown in FIGS. 1 and 2, by inclusion of a printing gap 42 in the liquid printing pattern in each layer along a bisecting line 101. The printing gap 42 is typically a portion of the printing pattern where no binding liquid is applied.

Forming a dividable tablet 10 includes forming a plurality of bound-together layers of a build powder. FIG. 6 shows a base layer 13 of bound powder, formed by printing a layer of build powder material with a binding liquid, thereby bonding the powder material in the printed areas into a bound-powder layer. A printing nozzle prints a multiplicity of droplets accurately according to a pre-determined (designed) printing pattern, with high precision and resolution within selected areas or regions of the surface of the powder layer, and at a volumetric or mass rate sufficient to wet the particles of powder material in the printed areas of the powder layer.

In the illustrated embodiment, the printing pattern 40 used to form the base layer of bound powder is illustrated in FIG. 8. The two dark (and darkest) areas are in the shape of semi-circles 41 and 41′, including chord edges 43,43′. The dark filling of the dark area indicates that the binding liquid applied by the print nozzles, under the control of a printing program and controller, is 100% of a pre-determined saturation level. In some embodiments, the 100% saturation level corresponds to a quantity of binding liquid of typically about 25 to about 35 mg per square centimeter and per millimeter thickness (mg/cm²/mm) of the build powder layer.

In other area(s) or region(s) within the dosage form, such as, in a non-limiting example, semi-circular print areas 48,48′ of the printing patterns 45,45′ in FIG. 9, binding liquid can be applied to interior portion(s) 30x of intermediate bound-powder layers 15x at a saturation level less than the 100% saturation level applied to semi-circles 41 and 41′ and the chord edges 43,43′ of base layer 13, but also greater than the 0% saturation level of the printing gap 42. In some embodiments, the quantity of binding liquid is about 6 to 25 mg/cm²/mm, more typically about 10 to about 25 mg/cm²/mm of the build powder layer.

Without being bound by a particular theory, it is believed that binding liquid applied onto a layer of powder material in these areas will migrate downward from the surface of the powder (where the liquid has been applied) through most or all of the thickness of the powder layer, thereby wetting most or substantially all of the powder material within the printed area. If a printed area(s) does not cover the entire upper surface of the powder layer, and there is one or more unprinted areas beyond the periphery of the printed area(s), the binding liquid deposited in the interior of the printed area will migrate downwardly (vertically) through powder beneath the printed area, while the binding liquid deposited at or near the periphery of the printed area will migrate both downwardly (by gravity and capillary action) through the powder beneath the periphery of the printed area and laterally (by capillary action) into the powder in the adjacent unprinted area. Once an upper surface of a powder layer has been wetted, and a next powder layer is deposited thereupon, it is believed that a portion of residual liquid at the upper surface of the first layer can migrate upwardly (by capillary action) and into the underside of the next powder layer, which is believed to ensure a bonding of the two successive powder layers into a unitary bound-powder structure.

The opposed chord edges 43,43′ of the respective semi-circles 41,41′ are parallel and spaced apart by the printing gap 42, where either no droplets of the binding liquid are deposited, or only a minimal quantity of droplets are deposited. Within the incremental powder layers, a width of the printing gap 42 is selected, and a volumetric or mass concentration of the binding liquid is applied along the chord edges 43,43′ of the respective semi-circles 41,41′, to allow at least a minimum amount (volume or mass) of the printed binding liquid that has been applied along the chord edges 43,43′ to migrate laterally into the zone of powder within the printing gap where binding liquid has not been printed. Without being bound to any particular theory, the level of lateral migration of the binding liquid into and through the powder material, coupled with any solvent vapor exposure during the drying step, is sufficient to bind together the particles of the powder layer within the zone of the printing gap, providing bound powder in the zone of the printing gap with a break strength sufficient to bind together the at least two sub-dosage units into a tablet that remains bound together under ordinary handling conditions, until a user manually divides the tablet along the functional seam. Determining the precise amount of binding liquid that migrates laterally toward or into the unprinted zone of powder is impractical, and not readily determinable.

The width or lateral dimension of the functional seam 20 is typically about 100 μm to about 500 μm, though preferably about 200 μm to about 400 μm, although the precise boundaries between the confront surfaces of a boundary wall and the functional seam 20 can be difficult to determine accurately. In various embodiments, the narrower width of a functional seam is preferred, provided its break strength is sufficiently high to secure or connect together the two sub-dosage units of the tablet, in order to minimize the loss of powder along the functional seam when the tablet is divided, and to promote dividing the dividable tablet into two or more substantially equivalent subunits along the functional seam. However, the functional seam width must also be adequate to ensure the two dosage form subunits do not fuse together excessively in one or more regions of the functional seam, which might lead to variability in the effective fracture plane when dividing the dosage form as intended.

In various embodiments, after the dividable tablet has been divided, the divided subunits have equal mass with a difference between the masses of the two sub-dosage units being about 3% or less, or being about 2% or less, 1.0% or less, or about 0.5% or less, and the amount of powder material of the dividable tablet that is separated or lost after the dividable tablet has been divided is less than 3%, which can be less than 2%, less than 1.0%, less than 0.5%, less than 0.3%, and less than 0.1%, of the total mass of the undivided tablet.

In some embodiments, the width of the printing gap can be widened by printing or dispensing a grayscale saturation level of binding liquid within the zone of the printing gap. The grayscale saturation level of binding liquid would be expected to be less than about 50%, more typically at least 10%, and/or up to about 30%, and including about 10% to about 25%. While effective in forming a dividable tablet, the broadening of the width of the printing gap to include a grayscale printing pattern may result in the bound powder matrix within the functional seam being less tenacious, resulting in the spalling (or falling away) of particles of the powder material from and along the functional seam.

Without being bound by any particular theory, the relative saturation level of binding liquid within the powder disposed in the zone of the printing gap, to form the functional seam, is about 10% to about 75%, more particularly about 10% to about 25% saturation. Typically, the saturation level of the bound powder matrix of the functional seam is uniform through the entire span of the functional seam.

A preferred embodiment provides a dividable tablet that divides into two (or more) sub-dosage units of equal weight and dosage amount, and with minimal particle loss along the functional seam. It is believed that a method that minimizes the printing gap optimizes the seam-breaking and tablet-dividing results.

FIG. 6 illustrates the manufacturing of a dividable tablet as an incremental series of intermediate layers of bound powder formed by printing of the intermediate layers of powder material with a binding liquid using a printing nozzle that prints a multiplicity of droplets accurately according to a printing pattern, with high precision and resolution at selected areas or regions of the surface of the powder layer, and at a selected volumetric or mass rate sufficient to wet the particles of powder material in the printed areas of the powder layer, thereby bonding the powder material in the printed areas into the intermediate bound-powder layers. In the illustrated embodiment, six intermediate bound-powder layers (15a-15f) have been formed using the same printing pattern 44 shown in FIG. 9. The printing pattern 44 includes two opposed, semi-circular printing patterns 45,45′. Each of the semi-circular printing patterns 45,45′ respectively includes a peripheral print area that includes an outer peripheral print area (47,47′ respectively) and a chord edge print area 46,46′. The dark areas, as described above, indicate that the binding liquid is applied by the print nozzles, under the control of a printing program and controller, preferably at a 100% or full printing pattern, to deliver a 100% saturation level of binding liquid.

In the interior, semi-circular print areas 48,48′ of the printing patterns 45,45′, any quantity of binding liquid can be applied, or no binding liquid is applied, to the powder layers. The powder within these interior portions 30x,30x′ of the intermediate layers are entirely enclosed within a surrounding or enveloping wall of a strongly bound powder matrix, namely, the top wall 16, the base wall 17, the boundary wall portions 36,36′, and the peripheral wall portions 38,38′, which allows the powder within these interior portions 30x,30x′ to be unprinted, printed with a grayscale printing pattern (less than 100% saturation), or printed with a 100% printing pattern. Preferably, interior portions 30x,30x′ can be printed with a grayscale pattern of about 20% to about 40%, and more preferably about 25% to about 35%.

In a non-limiting example, an intermediate bound-powder layer 15x can be printed according to printing pattern 45 of FIG. 9 at a 30% grayscale printing level relative to a base layer 13 and top layer 14 printed according to printing pattern 40 of FIG. 8 at a 100% printing level. The intermediate bound-layer 15x can have a liquid-to-powder weight ratio of about 0.10 to about 0.20, preferably about 0.13 to about 0.17, and a mass saturation of about 6.2 mg/cm²/mm to about 12.3 mg/cm²/mm, preferably about 8.0 mg/cm²/mm to about 10.4 mg/cm²/mm. Accordingly, the resulting tablet can have an average liquid-to-powder ratio of about 0.25 to about 0.45, preferably about 0.30 to about 0.40, and a mass saturation of about 15 mg/cm²/mm to about 28 mg/cm²/mm, preferably about 18 mg/cm²/mm to about 25 mg/cm²/mm.

Again, without being bound by any particular theory, when printed with grayscale or 100% (full) saturation level, it is believed that the binding liquid applied onto a layer of powder material in these areas can migrate downward from the surface of the powder where the liquid has been applied, and through the thickness of the powder layer, thereby wetting some, most, or all of the powder material in these printed areas.

Typically, after a suitable threshold level for binding is achieved, it is preferred to minimize the amount of binding liquid applied to a powder layer, if only to avoid having to evaporate or dry excessive amounts of solvents that remain after the forming of the bound powder matrices. In such situations, the break strength of the bound powder matrix within the interior contained areas 30,31, formed by the semi-circular printing areas 48,48′, is lower than the break strength of the bound powder matrix that forms the opposing boundary walls 36, 36′, and lower than the break strength of the bound powder matrix that forms the peripheral wall portions 38,38′, top wall 16, and base wall 17.

To complete the dividable tablet 10, as shown in FIG. 5 and FIG. 6, one or more top layers of bound powder is formed by printing a layer of powder material with a binding liquid using a printing nozzle that prints a multiplicity of droplets accurately according to a printing pattern, with high precision and resolution at selected areas or regions of the surface of the powder layer, and at a volumetric or mass rate sufficient to wet the particles of powder material in the printed areas of the powder layer, thereby bonding the powder material in the printed areas into a bound-powder layer. In the illustrated embodiment, the printing pattern 40, shown in FIG. 8, which was used to print the base layer 13, is used to print the top layer 14 of powder to form the top wall 16 of bound powder. The two dark areas are in the shape of semi-circles 41 and 41′, including chord edges 43,43′. The dark filling of the dark area indicates that the binding liquid is applied by the print nozzles, under the control of a printing program and controller, preferably at full saturation level. The binding liquid that is applied onto a layer of powder material in these areas will migrate downward from the surface of the powder (where the binding liquid has been applied) through the thickness of the powder layer, thereby wetting most or substantially all of the powder material within and beneath the printed area. Typically, binding liquid will migrate through most of or the entire thickness of the printed powder layer. Typically, the width of the printing gap 42 of the top layer printing pattern 40 is the same or similar to that of the intermediate layer printing pattern 44, and of the pattern used for printing the base layer 13.

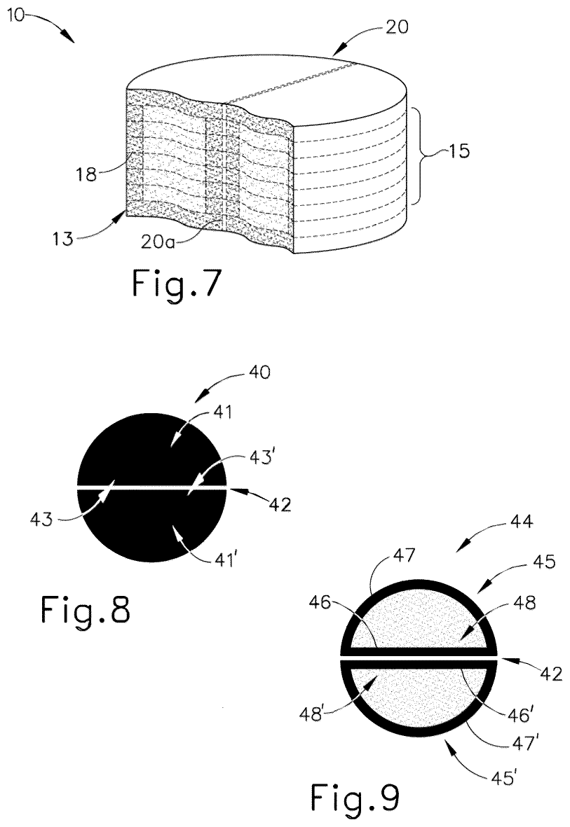

FIG. 7 is a perspective view of the tablet with an irregular segment removed from the sub-dosage units 12 and 12′ along and perpendicular to the functional seam 20, to reveal the interior portion of each sub-dosage unit 12 and 12′.

Later, after any excess binding liquid has been evaporated (naturally or by a drying process) from the wetted powder, a stable, bound powder matrix results. The bound powder matrix in the areas where a programmed 100% or full saturation of the binding liquid has been applied will have a break strength that will resist breakage of the bound powder matrix in such areas under ordinary handling conditions of the tablet by users.

### <Porosity>

For a given build powder composition and binding liquid composition, the saturation level of the binding liquid correlates with the break strength of the resulting porous bound powder matrix, and inversely with the porosity of the bound powder matrix. Consequently, an estimate of relative porosity of a bound powder matrix or portion thereof can be used to predict or estimate the relative break strength of the bound powder matrix or the portion thereof.

The bound powder matrix includes bound powder matrix portions of a functional seam 20, a boundary wall(s) 36, a peripheral wall 38, a base wall 17, a top wall 16, and an interior portion(s) 30x, each individually having a porosity from about 20% to about 90%. The boundary walls 36 have a porosity of about 25% to about 90%, which can be at least 40%, or at least 50% and up to 70%. The functional seam 20 has a porosity of about 20% to about 50%, which can be at least 25%, or 35%, and up to about 50%. The porosity of the functional seam 20 is higher than the porosity of the boundary walls 36, and is at least 5% higher, which can be at least 10%, or at least 15%, or at least 20% higher, or up to about 50% higher, which can be up to 40%, or up to 30%, or up to 40% higher, than the porosity of the boundary walls 36; for example, about 15% to about 25% higher. Without being bound by any particular theory, the higher porosity of the bound powder matrix portion of the functional seam, as compared to that of the boundary wall, results in a reduced break strength of the bound powder matrix portion of the functional seam, as compared to the break strength of the boundary walls. Consequently, the higher break strength of the bound powder matrix portion of the opposed boundary walls causes the bound powder matrix of the functional seam to fail and break when a bending force is applied to the dividable tablet across the functional seam, dividing the tablet into the two sub-dosage units.

The peripheral wall 38 has a porosity of about 20% to about 70%, which can be at least 30%, or at last 40% and up to 60%. The porosity of the peripheral wall 38 is at least 5% lower, and more typically at least 10%, or at least 20%, or at least 30%, lower than the porosity of the functional scam 20. The peripheral wall 38 typically as a porosity that is lower than the other portions of the bound powder matrix, to maintain the integrity of the sub-dosage units before and after division under ordinary handling conditions.

In various embodiments, the break strength, or hardness, of a bound powder matrix is proportional to the concentration of a binder material and/or binding liquid within the bound powder matrix. The content of binder material within the bound powder matrix can be introduced within the powder material that is deposited and layered, or within the binding liquid composition, or both. Other factors can also contribute to the break strength, including though not limited to the particle size distribution of the powder material, the powder composition, and the print image.

Although the illustration of the dividable tablet 10 shown in FIG. 5 uses certain light, dashed lines to illustrate the borders of different bound-powder portions of the bound-powder layers, the visual appearance of the outer surfaces of an actual dividable tablet will appear approximately uniform. For example, the zones of a bound powder matrix that have been printed with a 100% saturation and with a 50% saturation can, and often do, appear the same or similar to the naked eye after excess solvent has been evaporated away.

FIG. 10 illustrates a dividable tablet 210 that has been divided along the functional seam such as seam 20 illustrated in FIG. 5, into two substantially equal sub-dosage halves 11,1l′. The tablet 210 can be divided along the functional seam 20 by grasping the sub-dosage units 12,12′ of the tablet 210 between the fingers on opposite sides of the functional seam 20 and applying opposing forces (arrows marked “T”) to the sub-dosage units 12,12′ until the dividing functional seam 20 breaks. The subdivided tablet 210 consisting of the resulting tablet halves 11,11′ will typically include some minimum residue 23 of the functional seam 20 attached to each of the sub-dosage units 12,12′, and a minimum amount of lost powder that may flake off and fall away due to dividing.

FIG. 11 illustrates another embodiment of the invention wherein a physical score line is formed along the surface of the top layer 114, or the base layer 113, or both, with the physical score line extending in the same plane and parallel with the functional seam 20. The physical score line 122 assists the user in identifying the position and orientation of the functional seam 20. In a typical embodiment, the appearance of the bound powder matrix at the top surface of the sub-dosage units may appear identical to, or indistinguishable from, the appearance of the bound powder matrix at the top surface of the functional seam, and the user may not be able to see or discern the functional seam, and may be uncertain of where to grasp the tablet 10 correctly for dividing. In the illustrated embodiment, the top layer 114 of bound powder is formed using a printing pattern 140 for the binding liquid as shown in FIG. 12, which has a wider printing gap 142 as compared to the printing gap 42 (shown in FIG. 9) used in the printing of the intermediate layers 15. In some embodiments, one or more of the uppermost intermediate layers 15 may also be printed with a wider printing gap, to ensure that a row of unbound powder material is left unwetted by the printed binding liquid, which when removed leaves a shallow, physical line, illustrated as a gulley 122, that extends along the functional seam 120 and bisects the top layers 11 of the tablet 110.

Without limitation to a particular theory, it is believed that the comparatively large dimensions and porosity of dividable dosage forms in the instant application (as compared to conventional compressed tablets), in addition to other structural features help facilitate manual splitting and general handing by diverse groups of users, including without limitation geriatric patients or patients with limited manual dexterity for medical or other reasons.

### Dosage Form Characterization

The following procedures are used to characterize any of the tablets described herein.

### Surface Texture

The tablets can be inspected visually with or without the aid of a microscope. The texture of the tablets is generally analyzed to determine if the surface is rough or smooth and whether the edges on the upper surface and edges of the tablet perimeter are clean and sharp or rough and jagged.

### Hardness

Tablets are analyzed for overall hardness as determined by a tablet breaking force assay according to USP <127> using a VK 200 tablet hardness tester (Varian, US). The strength or hardness of the tablets is measured by a fracture test. A tablet is centered between the jaws of the tester and force is applied until the tablet fractures. The load at fracture is returned in kiloponds (kp). A kilopond is a metric unit of force measurement with 1 kp being equivalent to 9.807 Newtons.

### Disintegration Time

Tablets are analyzed for disintegration time using a basket-rack assembly according to the procedure described in the USP <701>.

### Bulk Density

The bulk density of the tablet is determined by measuring the tablet's mass and dividing that value by the calculated volume of the tablet. The tablet volume is calculated by measuring its dimensions and using the proper mathematical formula according to the tablet shape. For example, for cylindrical tablets, the volume is calculated using the geometric formula, V=πr²h wherein r is the radius of the tablet and h is its height. Typically, a tablet weighing 320 mg, and having a height and diameter of 5.15 mm and 11.6 mm, respectively, has a volume of about 0.544 cm³ and a bulk density of 0.59 g/cm³.

### Porosity

A porosity for a tablet can be estimated as the volume of the solids of the particulate material within a tablet compared to the volume of the intact tablet volume. A tablet is pulverized into a fine particulate material and highly compacted, and the volume of the pulverized, compacted particulate material determined. Porosity is determined according to Formula I, below:

$$porosity = \frac{intact volume - particulate volume}{intact volume} * 100$$

As a non-limiting example, a tablet weighing 320 mg, and having a height and diameter of 5.15 mm and 11.6 mm, respectively, is determined to have an intact volume of 0.544 cm³ and a particulate volume of 0.211 cm³. Accordingly, the porosity of the tablet is approximately 61%.

Alternatively, the porosity of a tablet can be determined using mercury porosimetry, in which the porosity of a material is measured by applying controlled pressure to a sample immersed in mercury, which cannot otherwise penetrate pores because of its high contact angle. Volume and pore size distributions of the material can be determined from the amount of pressure required to intrude into the pores and analyzed using the Washburn equation. It is expected that a tablet's porosity determined by mercury porosimetry will coincide with the porosity calculated above based on the intact and pulverized tablet volumes.

### Color

The color of a formulation comprising droxidopa can be evaluated by dissolving the sample in 0.1 N hydrochloric acid and measuring the absorption spectrum of the droxidopa solution using a UV-Vis spectrophotometer. Without being limited by a particular theory, it is believed that since the chemical structure of droxidopa is nearly identical to levodopa, the absorption spectrum of droxidopa can be predicted from the absorption spectrum of levodopa under similar conditions (see, e.g., Madrakian, T., et al., “Simultaneous Derivative Spectrophotometric Determination of Levodopa and Carbidopa in Pharmaceutical Preparations” Bull. Korean Chem Soc. (2004) 25 (12): 1764-1768) Accordingly, pure droxidopa is expected to have an absorption maximum at approximately 280 nm, while as the compound oxidizes over time, produces a yellow solution initially having absorption bands at about 300 nm and about 440 nm, before developing a third absorption band at about 330 nm and a first derivative absorption maximum (¹D) at about 350 nm. Therefore, it is believed that the development of any of the above absorption bands, particularly the sample's ¹D at 350 nm, can be monitored over time to determine the extent of the conversion of droxidopa to colored pigments.

### EXAMPLES

The following examples illustrate embodiments of the invention. However, it is to be understood that the following are only exemplary or illustrative of the present invention, and are not intended to limit the invention in any way, and numerous modifications and alternative compositions, methods, and systems may be devised by those skilled in the art without departing from the spirit and scope of the present invention. All masses and levels of components or compounds in a composition expressed as a ratio or a percentage are by weight, unless specified otherwise.

### Example 1: Initial Preparation of 3DP Dosage Forms Comprising Droxidopa

The following process is used to prepare several three-dimensionally printed (3DP) dosage forms having a multi-layer, bound matrix comprising 100 milligrams of droxidopa. The components of the binding liquid and the build powder, and the descriptions of the resulting tablets produced therefrom, are provided below in Tables A and B:

TABLE A

| | mass % | | | | | |
|---|---|---|---|---|---|---|
| Build Powder | 1 | 2 | 3 | 4 | 5 | 6 |
| Droxidopa (milled) | 35.0 | | | 35.0 | 35.0 | 35.0 |
| Droxidopa (sieved) | | 35.0 | | | | |
| Droxidopa (milled with | | | 35.0 | | | |
| hydrophobic silica) | | | | | | |
| Citric Acid | | | | 0.16 | 1.0 | 2.0 |
| Mannitol | 47.9 | 47.9 | 47.9 | 47.7 | 46.9 | 45.9 |
| Microcrystalline Cellulose | 12.0 | 12.0 | 12.0 | 12.0 | 12.0 | 12.0 |
| Hydroxypropyl Cellulose | 4.6 | 4.6 | 4.6 | 4.6 | 4.6 | 4.6 |
| Colloidal Silicon Dioxide | 0.5 | 0.5 | 0.5 | 0.5 | 0.5 | 0.5 |

TABLE B

| | mass % | | | | | | |
|---|---|---|---|---|---|---|---|
| Binding liquid | A | B | C | D | E | F | G |
| Purified Water, USP | 71.5 | 71.3 | 71.1 | 71.4 | 71.3 | 73.4 | 79.2 |
| Isopropanol, USP | 12.3 | 12.3 | 12.3 | 12.3 | 12.3 | 12.3 | 12.3 |
| Polysorbate 20, NF | 1.9 | 1.9 | 1.9 | 1.9 | 1.9 | | 1.9 |
| Glycerin, USP (%) | 3.8 | 3.8 | 3.8 | 3.8 | 3.8 | 3.8 | 3.8 |
| Copovidone, NF (%) | 10.5 | 10.5 | 10.5 | 10.5 | 10.5 | 10.5 | |
| Butylated hydroxyanisole (BHA) | | .167 | | | | | |
| Citric Acid | | | 0.4 | | | | |
| Ethylenediaminetetraacetic acid (EDTA) | | | | 0.1 | 0.1 | | |
| Ascorbic Acid | | | | | 0.1 | | |
| Hydroxypropyl Cellulose (HPC) | | | | | | | 2.8 |

The build powder formulation 1 is referred to below as “Primary Blend”, and the binding liquid formulation A is referred to below as “Primary Liquid”.

Build powder is prepared by first blending droxidopa and silicon dioxide and processing the blended mixture to deconstruct any droxidopa/silicon dioxide agglomerates that are present. The remainder of the components are blended into the processed droxidopa/silicon dioxide mixture, and the resulting composition is milled to deconstruct any agglomerates that formed. In build powder 1, the droxidopa/silicon dioxide mixture is milled. In build powder 2, the droxidopa/silicon dioxide mixture is sieved with 60 mesh. In build powder 3, droxidopa is milled with a functionalized silicon dioxide having hydrophobic moieties chemically bonded to its surface. In build powders 4-6, a droxidopa/silicon dioxide/citric acid mixture is blended together and milled, prior to blending the remaining ingredients and the final milling step.

The descriptions of the resulting tablets produced therefrom are provided below in Table C. Generally, the 3DP tablets are prepared by spreading an incremental layer of build powder of predetermined thickness onto a prior layer of powder, and applying a binding liquid to the incremental layer as droplets to bind the particles therein. This two-step process is continued until a printed dosage form consisting of a wetted powder matrix is formed, and, following separation of the printed dosage form and drying, a resulting orodispersible bound-powder 3DP tablet is formed comprising the target mass of droxidopa (100 mg).

TABLE C

| | | Tablet formulations | |
|---|---|---|---|
| | | Build | Printing |
| Formulation # | Description | Powder | Liquid |
| 1 | Primary Blend and Primary Liquid | 1 | A |
| 2 | Primary Blend and Primary Liquid + BHA | 1 | B |
| 3 | Primary Blend and Primary Liquid + Citric Acid | 1 | C |
| 4 | Primary Blend and Primary Liquid + EDTA | 1 | D |
| 5 | Primary Blend and Primary Liquid + EDTA/Ascorbic Acid | 1 | E |
| 6 | Primary Blend and Primary Liquid, no Polysorbate | 1 | F |
| 7 | Primary Blend and Primary Liquid + HPC, no Copovidone | 1 | G |
| 8 | Primary Blend and Primary Liquid, with Top Layer Dried | 1 | A |
| 9 | Primary Blend w/Sieved Drox. and Primary Liquid | 2 | A |
| 10 | Primary Blend w/Milled Drox. + Hydrophobic Silica, | 3 | A |
| | and Primary Liquid | | |
| 11 | Primary Blend w/0.16% Citric Acid and Primary Liquid | 4 | A |
| 12 | Primary Blend w/1% Citric Acid and Primary Liquid | 5 | A |
| 13 | Primary Blend w/2% Citric Acid and Primary Liquid | 6 | A |
| 14 | Primary Blend w/0.16% Citric Acid and Primary Liquid + | 4 | G |
| | HPC, no Copovidone | | |
| 15 | Primary Blend w/1% Citric Acid and Primary Liquid + | 5 | G |
| | HPC, no Copovidone | | |
| 16 | Primary Blend w/2% Citric Acid and Primary Liquid + | 6 | G |
| | HPC, no Copovidone | | |
| 17 | Primary Blend and Primary Liquid, at Full Tablet Dry | 1 | A |

FIG. 1 and FIG. 2 show perspective and cross-sectional views, respectively, of orodispersible tablets comprising any of formulations 1-17. Particularly, FIG. 2 illustrates sequentially formed incremental layers of bound powder, having a base layer 2, a top layer 3, and one or more intermediate layers 4. The exterior surface portions of the tablet 1 include the base layer 2, the top layer 3, and the peripheries 5 of the plurality of intermediate layers 4. The interior of the tablet 1 consists of respective interior portions 6 (inboard of the periphery 5) of the plurality of intermediate layers 4. The 3DP matrix structure forming the exterior surfaces of the tablet 1 has a higher hardness than the break strength of the interior portions 6, which is achieved by dispensing the binding liquid at a higher saturation level onto the build powder as compared to the interior portions 6.

FIGS. 3 and 4 show plan views of liquid printing patterns used to apply binding liquid on the layers of powder to form incrementally printed layers of the tablet 1 shown in FIG. 1 and FIG. 2. FIG. 3 depicts a solid printing pattern 7 used to create the base layer 2 and the top layer 3 of tablet 1, wherein a 100% or full saturation level of binding liquid is applied throughout the entire printing area, consisting of about 30 to about 32 mg/cm² of powder surface area, for each 1 mm of thickness of the layer of powder. FIG. 4 shows a second liquid printing pattern that includes two distinct printing regions, including an interior portion 8 and a peripheral portion 9. In the illustrated embodiment, the interior portion 8 of the printing pattern is a substantially uniform grayscale saturation level, consisting of about 10 to about 15 mg/cm²/mm. The peripheral portion 9 of the printing pattern comprises a substantially uniform, 100% saturation level of binding liquid used only in an outer ring of the printing area. The printing pattern shown in FIG. 4 is used to create the plurality of intermediate layers 4 of the tablet.

To assess the potential coloration of the tablets over time, tablets from each formulation are stored and conditioned within open blister packs (i.e., unprotected) under elevated heat and humidity conditions; particularly, at 75% relative humidity (RH) and either 40° C., 50° C., or 70° C. While conditioning temperatures of 50° C. and 70° C. are somewhat higher than temperatures typically experienced under ordinary handling conditions, they provide stressed conditions for assessing the effectiveness of the invention. At the time of manufacture, tablets of any formulation, including formulations described in further examples below, are typically white or off-white. The tablets are visually inspected at 1 day, 3 days, 7 days, 1 month, 3 months, 6 months, and 12 months from their respective initial dates of manufacture and conditioning.

Tablets stored at 40° C. generally retain their white color after one month regardless of their formulation. However, within 3 months many of the tablets begin to illustrate a pale brown tint, and after 6 months, all of the tablets illustrate some color change. Tablets containing citric acid improved resistance to color change, particularly tablets from formulations 3 (citric acid in the binding liquid), and 11 through 16 (citric acid in the build powder). Formulations 3 and 11-16 each exhibit an off-white color after 12 months, whereas the remaining tablets are medium to dark brown. Further, tablets in which citric acid is included in the build powder (formulations 11-16) appear to develop brown spots over time while tablets with citric acid in the binding liquid (formulation 3) do not, suggesting that the citric acid may not be completely dispersed throughout the entire tablet when citric acid is introduced via the build powder.

Tablets stored at 50° C. and 70° C. indicate a similar trend towards color-change resistance as tablets stored at 40° C., although tablets stored at the higher temperatures lose their white color more quickly over time. For example, after 3 months, tablets from formulations 3 and 11-16 stored at 50° C. look similar to their counterpart tablets stored at 40° C. for one year. The rate of coloration for tablets stored at 70° C. accelerates even further (off-white appearance after 1 week).

Without being limited by a particular theory, it is believed that the tested concentrations of BHA (binding liquid B), EDTA (binding liquid D and E), and ascorbic acid (binding liquid E) are all well below the IID (Inactive Ingredient Database) daily limit (mg) for each compound, and that increasing the concentration(s) of one or more of the compounds may have an effect on resisting color change over time. As a non-limiting example, while the concentration of EDTA utilized in binding liquid D is 0.1%, it was calculated that a tablet comprising a 100-mg dose of droxidopa can be formed from a binding liquid comprising as much as 0.2% of EDTA, assuming that the tablet is prescribed to be taken three times a day (as is common with droxidopa). Similar trends are also present for BHA and ascorbic acid as well.

Additionally, there appears to be no distinguishable color difference between any of the tablets in which the droxidopa and silicon dioxide mixtures were processed differently (formulations 1 and 9-10, corresponding to build powders 1-3) or in how the tablets were dried (formulations 8 and 17).

### Example 2: Assessment of Citric Acid Presence within the Build Powder

To further assess whether the method of introducing citric acid into the build powder affects the coloration of the 3DP resulting tablet, two additional sets of tablets are manufactured. Within a first build powder (build powder 7, below), the coarse citric acid utilized in build powders 4-6, above, is ground with a mortar and pestle prior to blending and milling with droxidopa, with subsequent blending and milling of the citric acid/droxidopa mixture with silicon dioxide. The particle size distribution of ground citric acid is examined using laser diffraction spectroscopy and is determined to have a D(10) of 7.81 μm, a D(50) of 102 μm, and a D(90) of 354 μm. For comparison, coarse citric acid is also examined by laser diffraction spectroscopy and determined to have a D(10) of 150 μm, a D(50) of 337 μm, and a D(90) of 584 μm.

Within a second build powder (build powder 8, below), coarse citric acid is dissolved in a solution of water and isopropanol, such that the concentrations of each component in solution are: water, 60% (w/w); isopropanol, 35% (w/w); and citric acid, 5% (w/w). The citric acid solution is sprayed as droplets onto droxidopa particles. The wet droxidopa/citric acid mixture is sieved to form granules and tray-dried. The granules are blended and milled with silicon dioxide. The remaining dry excipients are subsequently blended together.

Sets of tablets manufactured from build powders 7 and 8 are stored at 70° C. at 75% RH, and visually inspected at 2 days, 3 days, 5 days, and 9 days from their respective dates of manufacture. The components of build powders 7 and 8, and the descriptions of the resulting tablets produced therefrom, are provided below in Table D and E.

TABLE D

mass %

| Build Powder | 7 | 8 |
|---|---|---|
| Droxidopa (milled) | 35.0 | 35.0 |
| Citric Acid, coarse ground | 0.16 | |
| Citric Acid, granulated | | 0.16 |
| Mannitol | 47.7 | 47.7 |
| Microcrystalline Cellulose | 12.0 | 12.0 |
| Hydroxypropyl Cellulose | 4.6 | 4.6 |
| Colloidal Silicon Dioxide | 0.5 | 0.5 |

TABLE E

| | | Tablet formulations | |
|---|---|---|---|
| | | Build | Printing |
| Formulation # | Description | Powder | Liquid |
| 18 | Ground Citric Acid Blend + | 7 | A |
| | Primary Liquid | | |
| 19 | Droxidopa/Citric Acid Granule | 8 | A |
| | Blend + Primary Liquid | | |

Tablets from formulations 18 and 19 are visually inspected along with newly-printed tablets from formulation 6 as a comparison. After 9 days, tablets from formulations 18 and 19 have a consistent light brown color without visible spotting, and are noticeably lighter in color relative to tablets from formulation 6 (medium brown). Accordingly, without being limited by a particular theory, it is believed that the use of ground citric acid and pre-forming granules of droxidopa and citric acid each resulted in a more effective distribution of citric acid within the bound matrix of the resulting 3DP tablets.

### Example 3: Concentration Dependence of Citric Acid in Build Powder

To assess whether there is a concentration dependence for the ability of citric acid within the build powder to reduce coloration, four further build powders based on build powder 4 are generated (build powders 9-12, below), except that each further build powder comprises fine-grade, anhydrous citric acid at different concentrations. Five grams of build powder 9 and 2 grams of binding liquid A are blended together in a beaker and placed in a 60° C. oven for 60 minutes, resulting in the formation of white agglomerates (formulation 20). The process is repeated with build powders 10, 11, and 12 and binding liquid A, resulting in the formation of formulations 21, 22, and 23, respectively. Each formulation is placed in a capless 20-mL scintillation vial and stored in an incubator at 70° C. at 75 RH. Agglomerates are visually inspected 3 weeks from their formation. The components of build powders 9-12, and the descriptions of agglomerate formulations 20-23 produced therefrom, are provided below in Tables F and G.

TABLE F

mass %

| Build Powder | 9 | 10 | 11 | 12 |
|---|---|---|---|---|
| Droxidopa (milled) | 35.0 | 35.0 | 35.0 | 35.0 |
| Anhydrous Citric Acid, Fine | 0.08 | 0.16 | 0.32 | 0.64 |
| Mannitol | 47.82 | 47.74 | 47.58 | 47.26 |
| Microcrystalline Cellulose | 12.0 | 12.0 | 12.0 | 12.0 |
| Hydroxypropyl Cellulose | 4.6 | 4.6 | 4.6 | 4.6 |
| Colloidal Silicon Dioxide | 0.5 | 0.5 | 0.5 | 0.5 |

TABLE G

| | | Tablet formulations | |
|---|---|---|---|
| | | Build | Printing |
| Formulation # | Description | Powder | Liquid |
| 20 | Agglomerates of 0.08% Citric Acid Blond + Primary Liquid | 9 | A |
| 21 | Agglomerates of 0.16% Citric Acid Blend + Primary Liquid | 10 | A |
| 22 | Agglomerates of 0.32% Citric Acid Blend + Primary Liquid | 11 | A |
| 23 | Agglomerates of 0.64% Citric Acid Blend + Primary Liquid | 12 | A |

TABLE H

| | mass % | | | | | |
|---|---|---|---|---|---|---|
| Binding liquid | H | I | J | K | L | M |
| Purified Water, USP (%) | 75.3 | 82 | 85.8 | 83.9 | 77.2 | 87.7 |
| Isopropanol, USP (%) | 12.3 | 12.3 | 12.3 | 12.3 | 12.3 | 12.3 |
| Polysorbate 20, NF (%) | 1.9 | 1.9 | 1.9 | | | |
| Glycerin, USP | | 3.8 | | 3.8 | | |
| Copovidone, NF | 10.5 | | | | 10.5 | |

TABLE I

| | | Tablet formulations | |
|---|---|---|---|
| | | Build | Printing |
| Formulation # | Description | Powder | Liquid |
| 24 | Agglomerates of Primary Blend + No-glycerin Liquid | 1 | H |
| 25 | Agglomerates of Primary Blend + No-copovidone Liquid | 1 | I |
| 26 | Agglomerates of Primary Blend + No-glycerin, No-copovidone Liquid | 1 | J |
| 27 | Agglomerates of Primary Blend + No-Polysorbate, No-copovidone | 1 | K |
| | Liquid | | |
| 28 | Agglomerates of Primary Blend + No-Polysorbate, No-glycerin Liquid | 1 | L |
| 29 | Agglomerates of Primary Blend, Only Water and Isopropanol Liquid | 1 | M |

While each of formulations 20-23 exhibit a brown color after 3 weeks, there is an apparent concentration dependence between formulations 20 and formulation 21, and between formulation 21 and formulation 22, and their effect on the magnitude of the color change. In each instance, the formulation having the lowest concentration of citric acid demonstrates the greatest color change. However, there is no apparent visual difference between the color of formulation 22 and formulation 23. Without being limited by a particular theory, it is believed that tablets comprising any formulations 20-23 can be formed using any 3DP technique, particularly that of Example 1.

### Example 4: Effect of Non-Volatile Binding Liquid Components on Tablet Coloration

To assess whether any of the non-volatile components within the primary binding liquid (copovidone, Polysorbate 20, and glycerin) also affected the color of droxidopa tablets over time, several agglomerate mixtures are produced and stored under the same conditions according to the procedure described above in Example 3. The presence or absence of non-volatile binding liquid components is tested against the primary build powder blend (build powder 1). The components of the binding liquids, and the descriptions of the resulting agglomerate formulations produced therefrom, are provided above in Tables H and I.

To assess whether the addition of citric acid to the primary build powder blend may result in a further reduction in coloration of the droxidopa tablets, additional agglomerates based on build powder 7 and binding liquids H-M were generated and stored according to the procedures of Example 3 and above. Agglomerates resulting from the combination of build powder 7 and binding liquid F are also formed. Descriptions of the agglomerate products are provided in Table J.

TABLE J

| | | Tablet formulations | |
|---|---|---|---|
| | | Build | Printing |
| Formulation # | Description | Powder | Liquid |
| 30 | Agglomerates of 0.16% Citric Acid Blend + No-Polysorbate Liquid | 7 | F |
| 31 | Agglomerates of 0.16% Citric Acid Blend + No-glycerin Liquid | 7 | H |
| 32 | Agglomerates of 0.16% Citric Acid Blend + No-copovidone Liquid | 7 | I |
| 33 | Agglomerates of 0.16% Citric Acid Blend + No-glycerin, | 7 | J |
| | No-copovidone Liquid | | |
| 34 | Agglomerates of 0.16% Citric Acid Blend + No-Polysorbate, | 7 | K |
| | No-copovidone Liquid | | |
| 35 | Agglomerates of 0.16% Citric Acid Blend + No-Polysorbate, | 7 | L |
| | No-glycerin Liquid | | |
| 36 | Agglomerates of 0.16% Citric Acid Blend + Only Water and | 7 | M |
| | Isopropanol Liquid | | |

In addition to agglomerate formulations 30-36, agglomerates of formulation 18 are also produced as a control. A visual inspection after 3, 7, and 12 days indicates an apparent reduction in coloration in the vials containing formulations without glycerin compared to formulations that do contain glycerin. Further, each of formulations 30-36 exhibit less darkening relative to their counterparts in formulations 24-29, indicating the combination of no glycerin and the presence of citric acid in formulations 31, 33, and 35-36 demonstrate the least amount of darkening.

Because glycerin can be a versatile and useful compound for forming rapidly orodispersible tablets using binder jetting, several 3DP tablets are manufactured using a binding liquid containing glycerin at 0% (control, binding liquid H), 1.9% (binding liquid N, below), or 3.8% (binding liquid A), and both with and without citric acid, using the 3DP protocol described in Example 1 above. The tablets are stored within open blister packs at 70° C. and 75% RH and visually inspected at 3 days, 8 days, and 14 days from their respective dates of manufacture. The components of the binding liquid having 1.9% of glycerin, and the descriptions of the tablets produced herein, are provided below in Tables K and L.

TABLE K

| | mass % |
|---|---|
| Binding liquid | N |
| Purified Water, USP | 77.2 |
| Isopropanol, USP | 12.3 |
| Polysorbate 20, NF | 1.9 |
| Glycerin, USP | 1.9 |
| Copovidonc, NF | 10.5 |

TABLE L

| | | Tablet formulations | |
|---|---|---|---|
| | | Build | Printing |
| Formulation # | Description | Powder | Liquid |
| 37 | Sieved Droxidopa Blend + Primary Liquid | 2 | A |
| 38 | Sieved Droxidopa Blend + 1.9% glycerin Liquid | 2 | N |
| 39 | Sieved Droxidopa Blend + No-glycerin Liquid | 2 | H |
| 40 | Coarse Citric Acid Blend + Primary Liquid | 8 | A |
| 41 | Coarse Citric Acid Blend + 1.9% glycerin Liquid | 8 | N |
| 42 | Coarse Citric Acid Blend, No-glycerin Liquid | 8 | H |

As with the agglomerates formed above in Example 4, tablet formulations having reduced glycerin (e.g., formulations 38 and 39) or citric acid (e.g., formulation 40) each separately appear to demonstrate a reduction in color formation, while combinations of the two conditions (formulations 41 and 42) appear to reduce color formation even further relative to formulation 37. Without being limited by a particular theory, it is believed that while tablet formulation 42 (having citric acid but no glycerin) exhibits the best resistance to darkening, tablet formulation 41 (having citric acid but less glycerin than tablet formulation 40) is a non-limiting example of a tablet formulation that significantly resists coloration in the presence of glycerin.

### Example 5: Preparation of 3DP Dividable Tablets Comprising Droxidopa

Multi-layer 3DP tablets comprising either 100 mg, 400 mg, or 600 mg of droxidopa are manufactured. The ingredients for the printing and the build powder, as well as the relative component proportions and properties of the resulting dosage forms, are presented in Tables M, N and O1-O4.

Table M shows the ingredients and their mass percentages for the binding liquid.

Table N shows the ingredients and their mass percentages for the build powder.

Tables O1, O2 and O3 show the target mass, the actual mass and the mass percentages for the ingredients in the bound matrices for a 100-mg tablet (Table O1), a 400-mg tablet (Table O2) and a 600-mg tablet (Table O3).

TABLE M
Binding liquid

| | | Mass % |
|---|---|---|
| Ingredient | Manufacturer | (w/w) |
| Purified Water (USP) | | 73.0 |
| Isopropanol (USP) | | 12.3 |
| Copovidone, NF (Kollidon VA64) | BASF | 10.5 |
| Glycerin (100% Vegetable Base, USP) | Avantor | 1.9 |
| Polysorbato 20, NF | Penta | 1.9 |
| Anhydrous Citric Acid (Powder Grade, USP) | Avantor | 0.4 |

TABLE N
Build Powder

| Ingredient | Manufacturer | Mass % (w/w) |
|---|---|---|
| Droxidopa (milled) | MSN Labs | 35.0 |
| Mannitol (Pearlitol 160C, USP) | Roquette | 47.9 |
| Microcrystalline Cellulose | FMC/DuPont | 12.0 |
| (Avicel PH 101) | | |
| Hydroxypropyl Cellulose | Nippon Soda | 4.6 |
| (HPC-L-FP) | | |
| Colloidal Silicon Dioxide | Cabot | 0.5 |
| (Cab-O-Sil MSP) | | |

TABLE O1
Bound Matrix

| | 100 mg | | |
|---|---|---|---|
| | Target | Actual | |
| | Mass | Mass | Mass % |
| Ingredient | (mg) | (mg) | (w/w) |
| Droxidopa | 100 | 104.1 | 33.2 |
| Mannitol | 137 | 142.6 | 45.4 |
| Microcrystalline Cellulose | 34.3 | 35.7 | 11.4 |
| Hydroxypropyl Cellulose | 13.1 | 13.6 | 4.4 |
| Colloidal Silicon Dioxide | 1.43 | 1.5 | 0.47 |
| Copovidone | 11.3 | 12.6 | 3.8 |
| Glycerin | 2.05 | 2.3 | 0.68 |
| Polysorbate 20 | 2.05 | 2.3 | 0.68 |
| Citric Acid | 0.43 | 0.5 | 0.14 |
| Total (mg) | 302 | 317 | |

TABLE O2
Bound Matrix

| | 400 mg | | |
|---|---|---|---|
| | Target | Actual | |
| | Mass | Mass | Mass % |
| Ingredient | (mg) | (mg) | (w/w) |
| Droxidopa | 400 | 416.4 | 33.3 |
| Mannitol | 547 | 569.4 | 45.5 |
| Microcrystalline Cellulose | 137 | 142.6 | 11.4 |
| Hydroxypropyl Cellulose | 52.6 | 54.8 | 4.4 |
| Colloidal Silicon Dioxide | 5.71 | 5.9 | 0.48 |
| Copovidone | 41.9 | 43.6 | 3.5 |
| Glycerin | 7.59 | 7.9 | 0.63 |
| Polysorbate 20 | 7.59 | 7.9 | 0.63 |
| Citric Acid | 1.6 | 1.7 | 0.13 |
| Total (mg) | 1202 | 1251 | |

TABLE O3
Bound Matrix

| | 600 mg | | |
|---|---|---|---|
| | Target | Actual | |
| | Mass | Mass | Mass % |
| Ingredient | (mg) | (mg) | (w/w) |
| Droxidopa | 600 | 624.6 | 33.4 |
| Mannitol | 821 | 854.6 | 45.7 |
| Microcrystalline Cellulose | 206 | 214.4 | 11.4 |
| Hydroxypropyl Cellulose | 78.9 | 82.1 | 4.4 |
| Colloidal Silicon Dioxide | 8.57 | 8.9 | 0.48 |
| Copovidone | 59.3 | 64.6 | 3.3 |
| Glycerin | 10.7 | 11.7 | 0.6 |
| Polysorbate 20 | 10.7 | 11.7 | 0.6 |
| Citric Acid | 2.26 | 2.5 | 0.13 |
| Total (mg) | 1797 | 1865 | |

Table O4 shows various tablet properties, including the dimensions, density, porosity, and ratios of the binding liquid (fluid) applied to the build powder used for the tablet generally, for the boundary wall, and for the interior core.

TABLE O4
Tablet Properties

| | | | | | | | Fluid to | Fluid to | Fluid to |
|---|---|---|---|---|---|---|---|---|---|
| | Target | Actual | | | | | Powder | Powder | Powder |
| Droxidopa | Mass | Mass | Diameter | Height | Density | Porosity | Ratio | Ratio | Ratio |
| (mg) | (mg) | (mg) | (mm) | (mm) | (g/cm³) | (%) | (Tablet) | (Boundary) | (Core) |
| 100 | 302 | 317 | 11.2 | 5.15 | 0.584 | 61.1 | 0.38 | 0.51 | 0.15 |
| 400 | 1202 | 1251 | 20.4 | 6.33 | 0.598 | 60.0 | 0.35 | 0.53 | 0.16 |
| 600 | 1797 | 1865 | 23.4 | 7.11 | 0.601 | 59.6 | 0.33 | 0.52 | 0.16 |

Particularly, first and second layers of powder material, each having a substantially uniform thickness of about 400 microns, are deposited onto a printing bed. After each of the first and second powder layer is deposited, the printing bed is passed under a bank of liquid printing nozzles (Starfire 1024 LA, Fuji Dimatix), which dispense binding liquid in-sequence according to printing pattern 40, as illustrated in FIG. 8. The printing pattern 40 has a 100% saturation level of 31 mg/cm²/mm binding liquid. The two semi-circular patterns 41,41′ that have the same diameter and are bisected or divided by a 0.25-mm wide printing gap 42 where no binding liquid is dispensed. After binding liquid is dispensed, deposition of a successive powder layer is optionally delayed to facilitate partial drying of the printed layer before the next manufacturing step.

Several additional incremental layers are printed in alternating steps of depositing a powder material and dispensing binding liquid onto the deposited powder material in a printing pattern 44 as illustrated in FIG. 9. The 100-mg tablet is printed with 8 incremental layers, the 400-mg tablet is printed with 11 incremental layers, and the 600-mg tablet is printed with 13 incremental layers. Peripheral patterns 47,47′ and respective boundary wall patterns 46,46′ are printed with binding liquid at a 100% saturation level, while respective interior portions 48,48′ are printed at a 30% saturation level. The two semi-circular patterns 45,45′ are separated by the same 0.25-mm wide printing gap between the respective boundary wall patterns 46,46′, which register over the printing gap of the previous incremental layer. Binding liquid dispensed in the boundary wall patterns 46,46′ form respective boundary walls consisting of a bound powder matrix.

Penultimate and final layers of powder material are successively deposited and binding liquid sprayed onto the layer according to the same procedure, printing pattern, saturation level, and printing gap as the first and second layers above. The completed tablet has a functional seam that is sufficiently durable for the tablet to be maintained as a single unit under ordinary handling conditions, but sufficiently frangible to allow a user to break the tablet along the functional seam to form two substantially identical subunits with minimal loss of mass.

### Example 6: Preparation of Dividable 3DP Tablets with a Physical Score Line

Multi-layer 3DP dosage forms having a physical score line are manufactured as dividable tablets using substantially the same process as described above in Example 5, except that after each of the penultimate and final layers of powder material are deposited, binding liquid is sprayed with a printing pattern 140 as illustrated in FIG. 12. The utilization of a printing gap 142 that is wider than the printing gap 42 above forms a center portion of unbound powder material atop each of the top two layers. After the unbound powder material is removed from the printing gap 142, it leaves a gully in the top surface of the dividable tablet that indicates to a user where the tablet should be divided into two functionally equivalent tablet halves.

### Example 7: Preparation of Additional 3DP Dosage Forms Comprising Droxidopa

Any of the 3DP processes described in the examples above are used to prepare three-dimensionally printed (3DP) dosage forms having a multi-layer, bound matrix comprising droxidopa. As a non-limiting example shown in Table P, resulting tablets can comprise the following ingredients.

TABLE P

| Tablet 1 Ingredient | Mass (%) |
|---|---|
| Droxidopa | 30-35 |
| Citric Acid | Up to 3 |
| Mannitol | 40-50 |
| Microcrystalline Cellulose | 5-15 |
| Hydroxypropyl Cellulose | 3-7 |
| Colloidal Silicon Dioxide | 0-2 |
| Glycerin | 0-2 |
| Copovidone | 3-7 |
| Polysorbate 20 | 0-2 |

In another non-limiting example, some of the resulting tablets comprise the following ingredients in Table Q

TABLE Q

| | Bound Matrix | | | | | | | | | | | |
|---|---|---|---|---|---|---|---|---|---|---|---|---|
| | Tablet 2 | | Tablet 3 | | Tablet 4 | | Tablet 5 | | Tablet 6 | | Tablet 7 | |
| Ingredient (% w/w) | Low | High | Low | High | Low | High | Low | High | Low | High | Low | High |
| Droxidopa | 1 | 10 | 10 | 20 | 20 | 30 | 35 | 40 | 40 | 50 | 50 | 60 |
| Mannitol | 61 | 78 | 51 | 69 | 41 | 59 | 26 | 49 | 16 | 44 | 6 | 34 |
| Microcrystalline Cellulose | 10 | 15 | 10 | 15 | 10 | 15 | 10 | 15 | 10 | 15 | 10 | 15 |
| Hydroxypropyl Cellulose | 3 | 7 | 3 | 7 | 3 | 7 | 3 | 7 | 3 | 7 | 3 | 7 |
| Colloidal Silicon Dioxide | 0 | 1 | 0 | 1 | 0 | 1 | 0 | 1 | 0 | 1 | 0 | 1 |
| Copovidone | 3 | 5 | 3 | 5 | 3 | 5 | 3 | 5 | 3 | 5 | 3 | 5 |
| Glycerin | 0 | 2 | 0 | 2 | 0 | 2 | 0 | 2 | 0 | 2 | 0 | 2 |
| Polysorbate 20 | 0 | 2 | 0 | 2 | 0 | 2 | 0 | 2 | 0 | 2 | 0 | 2 |
| Citric Acid | 0 | 2 | 0 | 2 | 0 | 2 | 0 | 2 | 0 | 2 | 0 | 2 |

It is well within the knowledge of persons of ordinary skill in the art to devise formulations of build powders and binding liquids that can result in 3DP tablets having the above ingredients based on factors including, but not limited to particular 3DP additive manufacturing method(s), available 3DP equipment, and desired properties in the resulting tablet(s). Any tablet having any of the ingredient(s) and concentration(s) in the formulations listed above can also further comprise additional excipients, including but not limited to one or more binders, one or more disintegrants, one or more dispersants, one or more sweeteners, one or more glidants, one or more flavorants, one or more surfactants, one or more humectants, one or more preservatives, one or more antioxidants, one or more fillers, and one or more diluents. Non-limiting examples of such additional excipients that can also be comprised within any of the formulations above are ascorbic acid, BHA, BHT, sodium metabisulfite, and EDTA. Without being limited by a particular theory, it is believed that mannitol can be utilized to balance the mass of the tablet once the desired masses of the droxidopa and remaining excipients are selected.

Additionally, one or more of the ingredients in the tablet formulations above can be substituted or combined with another compound having substantially the same structure and function, while maintaining the concentration(s) indicated above. In one non-limiting example, the 0-2% of citric acid in any of tablets 2-7 can be substituted, or combined, with one or more acidulant compounds selected from the group consisting of succinic acid, tartaric acid, oxalic acid, and phosphoric acid, so long as acidulant's total mass of 0-2% within the tablet are maintained. In another non-limiting example, the 3-5% of copovidone in any of tablets 2-7 can be substituted, or combined, with povidone, so long as a total mass of 3-5% of the compound(s) within the tablet are maintained. In another non-limiting example, the 3-7% of hydroxypropyl cellulose in any of tablets 2-7 can be substituted, or combined, with hydroxypropyl methylcellulose, so long as the total mass of 3-7% of the compound(s) within the tablet are maintained.

While particular embodiments of the invention have been illustrated and described herein, such details are not intended to restrict or limit the scope of the appended claims. Accordingly, while some embodiments are particularly described and illustrated herein, it should be understood that additional modifications and variations of these embodiments, and the equivalents thereof, are within the scope of the invention as recited in the following claims.

1.-30. (canceled)

31. A rapidly orodispersible tablet comprising a porous, bound powder matrix comprising up to about 80% droxidopa, up to about 5% of an acidulant, and a binder, wherein the droxidopa comprises particles of the droxidopa bound within the bound powder matrix by the binder, and has a porosity of about 30% to about 80%.

32. The tablet according to claim 31, further comprising about 3% to about 35% of a disintegrant, wherein the bound powder matrix further comprises particles of the disintegrant.

33. The tablet according to claim 32, wherein the disintegrant is selected from the group consisting of microcrystalline cellulose, cross-linked polyvinylpyrrolidone, croscarmellose, sodium starch glycolate, and a combination thereof.

34. The tablet according to claim 32 wherein the bound powder matrix further comprises particles of the binder.

35. The tablet according to claim 31 wherein the bound powder matrix further comprises particles of the binder.

36. The tablet according to claim 31, wherein the tablet comprises up to about 2% citric acid.

37. The tablet according to claim 36, further comprising up to about 5% glycerin.

38. The tablet according to claim 37, wherein a mass ratio of the citric acid to the glycerin is from about 1:3 to about 1:6.

39. The tablet according to claim 31, wherein a portion of the acidulent comprises particles of the acidulent distributed throughout the bound powder matrix.

40. The tablet according to claim 31 further comprising up to about 5% glycerin.

41. The tablet according to claim 31, comprising 20% to 66% droxidopa.

42. The tablet according to claim 41, containing 1 mg to about 5,000 mg droxidopa.

43. The tablet according to claim 42, containing 100 mg to 1,800 mg droxidopa.

44. The tablet according to claim 31, wherein at least a portion of the acidulant is molecularly dispersed throughout the bound matrix.

45. The tablet according to claim 31, wherein the bound powder matrix comprises at least five layers of the bound powder matrix that are bonded together by the binder.

46. The tablet according to claim 45, wherein the bound powder matrix has a porosity of about 5% to about 80%.

47. The tablet according to claim 31, wherein the bound powder matrix has a hardness of at least 1 kp and disperses in 30 seconds or less in a volume of 30 ml or less of water or saliva.

48. The tablet according to claim 47, wherein the citric acid particles have a D(90) value of 75 microns or less.

49. The tablet according to claim 48 further comprising up to about 5% glycerin.

50. The tablet according to claim 49, wherein a mass ratio of the citric acid to the glycerin is from about 1:3 to about 1:6.
